SEQUENCE LISTING (22 sequences)

SEQ ID NO: 1 - DNA, 13521 bp, Spinacia tetrandra
  misc_feature 1..13521: note = first genomic fragment genomic DNA
  source 1..13521: mol_type = genomic DNA
atagtcaaagtagattatataaatagtggcaaaagtccaaacgttgcgagtattaaaagacggaggaagtatatagtttcaaaagaaaacttacatattgcaaatgaggaaattcaagttctgctatataatttagtctactaatagtgtgtttatgttacaaaatctctaattcccaattttaatatgtgacacgtcaatatttacaacacaaataatttgtcgcaagttacgacctttatcactttgctacacaaaatgacctagaaattatatcaatcaagcccctatacagtatacaccaattgaaatctaaaaaattacatgatctagggtgcaactctgttacataaacaccatttactgaactactcctgtttggcaaatgacaaatccaaggggggaaaaatcactttttccaagaaaattagttttgaaaaatgtggatttccatggaagaagtaaattttccttggatttatcacctcccctacggctccgattcatcatctgatacttgactattagttgtgctccactcccgaagcatatcgggtagactttggctatctgtacaaaatgcaggctgcaaaggtgcttcaagggtgaccgaattcgtactcaacataacatgaacctcagacatagttggcctgtctgcagggctttcttggacacacaacaaaccgatatggatgcatcgcagtatctcggttctagagctgcctagtaacacaggatctaccagattccaagctgtgccttcattccagtttctccatgcctatacacaaatgtcatccatgaatgaggtcaagtttgtcaattttttgcaacgtaaatttagaattaaccaatcagataatcaaacttacagaggtcgtgaggttttctccctcgtaatgaaatgaagtaatcctctgcccgcttactatctctaggactagtataccaaagctgtatacgtctgactttactgatacttgatattgtgtagcatactcaggtgcaatatatcctctgaataacaaaaatcgtttctcaaaataagaaaacaaaagcatacagagtattagacattatgtaattaactaaatataaagttccgatgcttacaaggttccagcgattctgctagcctttgtttgagtttgatctatgttaaatagccatgccataccaaaatctgcaatcttgggacaaaaatcttcatcaagtagaacacttcctgcttctaggttgcgatgtatgatcattagtctggaaccttcatggaggtatagaatccctctagctatgccattgataatcttataacgaatttcccattgcatagactgacgactcaatggatctgcattttattaccaaaatagtatgatggacactggatacatgtatcatgcaatgaaactagtccttagagtcgtaaacaaatctaatatacatcataatcatttgaaaatcacccggattggattgttatacatcccggtagtatgtgtaaaatctcgaattgtagtagtgattacataccaaatataaagttgtcaaggcttctgttggcaacaaactcatagacaagaatcctttcttctccatgcaagcaaaatcctagaagtttcaccaaattcttgtgccggaccttggctaatatcaaaatttcatttttgaactctgcatctctttggcgcgagttccttaaaagcctcttaacttctatctcttgcccatcaggaagtataccctacaaagaacaaataagctggttaatcatttgaagtaatacactaatacgtagtacaccagtactatattaactgaattagtaccttataaaccgggccgatgccttctccgatcttattatcatcagaaaagttatgtgttgcaatttctatagtttcaaggtcaaattgcaaggattggagggactcaacttcgtcttctgcacttataccatgaacaaagttatacttgtagatagttaggagctactaggtatatataaaaatatgtaaccttttacacacacacaataaaaaagaagatcttactggtaaaagaatgattcagatttctgatttcctgtagttgggagtttgattcttccagtttcggacagagaggaaaatttctcaaattatcacaaaatgtaattaacagatgagaaaggtgatggaacgaatatgaatgtgtttgaaaccctgctacttctcgtgtctcacccatatctgattctaatttccaccatccttccaactttggcatactccaaagctcaagcttttcaagggatggaaagaaaaccaagtcctcatctgctcctccatgacttgaccctcctgatgtcgatgcaaccccttctgcgctgataatactattctccatatacaccacttcactcaaatatcgtaatgtaaggaatttcaaatgacgcagttgactcatcaatggaagatgctccaaccttgtacatccgtatagtgttatgctaacaagatttgggagacaagtatggagattcattgatgacctcccccagcttgggaacctaattcctggataatccctcaactccaacattcggagattaagatggggttgcagcccttgcagcaccacatctgcatttttcccttgcagcaatgcatctgcatcagtcccctcactctcatgattccaaacatactgaatttccctcaaatgttcactttttaatagaaaccccttttccctagcatcaactgcatcttttttgacataaattaccatattgtggctatgattgctgaaatttttcaaatctcccagctcaccaacactaactgacccccgattccagctatttctttttctgctcactacaaaccctgtcagtttatgcagagatgtcatgctattcatcccccggggcatatgcgacaaactcttacagccttgtatatctaggtgccttagatttactaatttcctcaaatcgcttggcaaagtttttagaccacgacactcacgtaacttcaatatttgcaagttgtaaagcttggtaatggaggtaggcaacttagagagacggtaattatctgataggtcaagatatcttagatgcaacaagttacctatctcactcggcaacatttccaaatcagagttattcaagcttaagacccttaaatattccacttttgataacaaatatgttgaattagaagaaagcgcatctattctaaaaattgaacgcatccttttcatattggtgaaatgacgataaatatctttaacggcatctctatcaataaatatatggcgagttttctcgctaaacccccttatattatacttggacgacacgatttctgttcctgctacttcactagcaaggtcatgaatcaagtcatgcatcttgcatgatagaatttcatcagtaccctcggcccgttctacatcttggaagaaacacctttggagtaattgtttgaaatattcctcacctgcattttcgaaactctcttcatttgatgagatgatgaagccttgtgccatccagagactgatcaaatcctccttgataatcttgtaatccttggggaatacagaacaatacttaaagcaactcttcaaatggaacggaagataataataactaatcttcaaaacaggtaaaataccattgatctcatcttgtggtatatttgccaagtcagtgtttttcaaatattgccacctactttcaccctgacctcgaagaagacttccaaccacccttatagcaagtggaacatttgcacactttctcacaatatcttttccaattttaaccaagtgctcttgcatttgatgttgtcctggttcaagtgtcatcctctcaaacaaatcccaagactctttattagagagtccttccaatttatgaatatgatagcttccaacaactttcgccaccccacttgagcgagtagtcaccagaattctgctaccgtttctacctccaatcaatagttttctcagctcatgccatttaatgggatcttcagtccatacatcatccaaaacaaggaggtatatcttcccatccagttcgtcccgaattttcatctgaagctggtccatactcatactcagggcatcgtgctttcttctagggtttacggctgcaaggatttcagcaacaagatctttgacattgaaactatcagagacacaaacccacattttcagagagaatttactacctatcttctcatcattataaacaagtcgggctagagttgtttttcccagccccccaatccccactatgctgacaacagaaatatccttctcctcacaagaagtgtttagtaccatatctatcactttccgcttgtcatcatccctcccaatgatcatatcttcctcgcaaacaaaggaatgagtttcccttgctttgattctcagctgtttcgcatccagactaacggggtgtctggagccaaagtcatagcgatgatctttgacaatatcattcaacatctccctaatgttctccatctcacgagacccagtgaaagcagaacgaagcttattagaggaagagaagaaaaggcatacctcattaagcagcttgttattaaagcgcattatgagggttttgcgttgagcaagagtggcaatcttgtcgaacaagtcatcagctatataaagagcttccttaagcctctgaagcctatccagttcaacatggctacgacgatattgatgcaactgctcttgatcttcagcatcaaggagcacagcctcaatagtggacatggtggccttgagtttgtcaagctgtgagtactccacacccaacaaagattgaatctgtgtccaaatgggagaacccaacaattcgaggacagtttgtactagtgaaaggcctgcagacacatccatctctctctcttttgttatgaattagtattgcttattttgaggaatgcaaaggaaataatgaagtgagttttaaaattcctctactccgttataaataaaggcatatacttgtattgtgtttgggctggaagaacgtcttacaattcgaggaaagagtgaggaaagagtgagaaagcaaagacattgtcttgtccaaagtatgtacaacagtacaagtgattttcaaggttagttggtagacccacaagtaaataacatatgacaaacttgcttaagccacatttttcttcgtttacttgagttgacttgacatattgcaactttcctacttgctgcatcaactatactgaaagggctcattttcttccacccatctcatctcaagcccaacttgacagcaacgttgattttcctcaacttttgtggaggataattgataatttaataccctctatccctatttgttttccccatattttattttggtccatcctttaaaaattgtccggtttctaatttccttatatagtaacatgtccccaccatttatcccacgtgtatacactcaaaaagtcaaaaagacatagatacccccaaattccttgtaaattttcggttcaacccttaataaagcacacattgtctacgaggcaagggacggagggtgtacgcaacctttcaagtaatacagagttaggtccaagtttaggatcaggtcaagtaaaacaactgtataaccatactcaacactgccccaccggagtcaagtcgaaataaccaagtctttgaaagagaagacatgacctaatgttcgggcccaacgcacaaaaacaatagagtagaaatgtacaattcatttccattgctgtgtagtggaaaatttgtcaagtacgacataggttgtttagaactttatcaattactactaactactccgtactcttcactgataagttgtcaattactaactaattactacataggttttgttaaaagttgtcaattattagctattttctagcaatagaggtcaattaatacgttttccgaccaattagtcgactaatcacatcataatcactttaattatactttaatgacccaaaattacaactatcaaacccagaattacaactatcaaaaccaaaactacaatataccttgagtagaagaatcagatcgaaacggatagctgattaaaatctattaacggacgctccacagatctctcgttgatttcaacccttgcattgcaaaaatcatcaaaagtggaatttaattttgcagaaatttaaaatagaagaggacatacagaaagcgtcgagaggagattggtcaagcttgcgcttaaccctaacgacgatagacttgttatcgccattgttgtcgcttgataagcttgaaattgaagaacttctcgagacttcactattcatatttcgctccattttttttgggtactcttcaagtttgtgttcaaagtggattaattttgcatgggttaagttaaaaaaaaagttggaaatggttcccgggttgttttcgtgttactccgtgctccgtaatgagcagtacccgggtcaccgggataggcacataagtggagcgggtttaatcggagacccgacccgtcttgcatccgccccaagtaggtggggacgggcaaggtttgacgagtgatgagtgggaacgccgaatgcatataaaaattgtacccattatgacttatgaatgatgggacgttgcggattttgtgttagacccgcccactttatacccgtcaacccgattcgaatattctttcttacgtgaattaaaacttttaaagaaaatctacaattaaacatttttattaatgtgttttttttccattatacttcgtatatgttagttttagttgtaaggtattttttaaaagtttcttcattatatatgttgtaaggtgtttgctaaataagaaaaaaaatagcagtattggcacgggtatgggtgggacgggatgaaatcggaatggataaccaagcgggtggtaaggcggggatggattttagtttgtacccgttggggaagagatgaggataaattttgtatccgccatggaggatggggcggaaatgggatgaaaatttaggtgggatggggatggaaggacctacatccgcatctgctccgccatttgcatccctaatatgggcagaccagatcaaatatttacaataaataattaatacatgcaatacttgtatcattattataagataatgaattcatcttcattaatctttgtgccggtcaacatgtaattcttataaaaaaaaaggagggattatattattcctttttctcccataaaaaattgtaatcccctgctatttcgttcatatttttggataaatgattggtctggtcgatactcgatcgagtagtagaaaaatatatctttttttattggagagatatcaggtttttcataaagttaagtcaattttcgcaaaaatcaaaatgttttgttaaaatgtgtcaatattatgattaattagattgttactacgtatataactggtcttgagtctcatactatgtgaatgggatagtatttaatgaattttactccgtataatgtaagaattaaaaacttgtaaaaagtattgtagtgagtctaatgactagctagtggtgtgagactatcgactaatatgagacatatcaattacatgttggttcagtgatgactggggttgaacttggtagggcgaaccgtgtgttcggtcccctacaataacaaatctactccaaaactcaccccgaatccaaattagcgttaaaaatggtaaaaaaatataaaaaatatgagacatacaaataaagggagattttgtttggaatgtttgatatttgtggcttgattaaaaaaccaggttcatgcttaaaaaaaaaaaaaaaaaaaaaaaaagatgaaccgaaaaaaacccgaagttgtcaattgtcatcctactgatcaaaaccacaaaactaaaatgaatcttcttgcagataacgtggcccattccagtaactcatgaattaaccaacttaatttctgggaatttttttaacttgatgacagaaaaatggcgttgattggcacccccctctctaaccccaaaacccaccaaattttctgtcaccaccatcattttcatcaaattaaacccttaatttctgttcaatttccccaatttcccaatacccatttgtctaatcacctcagaaaacccagcttaagatgtttttctcaaaaacaacgccaacccacccaaaattcactcaatcaagtagagaaagaggaagaagaagtaggaaaagaagttcaaaagtatgaatttgagaggttgttttcaaacattaatcaagccactttgaagcatgaacctggtttgaattttagcttaatttttctcatattgcttagttatagattggattttcattattttgaagtttgattgttttcttgattatttccttttcattttaccctgttggcaggtagtgttaccagttcaattttccttgtggcaggcactacggtatgctatagactatagttgccaattttagcttattgaatgaaatttgagctttttttagtgaaaattaagaatgttggattagttattagtgcacacctcttagttgtgtgtgtgtgagttgtgtgtgtgagtgtgtgtgagttgtgtgtgtgtgtgtgtgttttaatgctagtgaaagcacgaatgttgatcgtggacacggtatctgtattgcaattacatgttcagatttgggggattaatggagcttttacagtttttgaaaatggcattttgatcgcggtatactatattgcttttacatgttcaagttttacctcatagttatgggattaatgcaagtgaaagaggcaatgttagtacattccatgcctagggaaaatgtccggtcctgtaattaatgcaagtgtgattgtctaattctatggtgatgaatgatgattatttggaactttaacatttcaatgaagcatctgactgctggaagcattttgaaaatggcattttggttgcaaaattcgcgatatactatattgcttttacatgttctactttgttctttttgtatggtgtatatattgcttgctctactacatgttccctgatatgattagtttattatccatttttcactatgtttttcctttctgttcagttatgactcattttctgtgacaattttacagataggtgcaggtattttagcacttcctgctgtgacacaagaatctgggtttcttgcctctgctgtcgcgtgcattggttgttgggtatatatggtaagggcttggcactaaggtgttctaatatttattccccatagctatatagtttctttctgcctcttttacagcgaattcatgtatttgattgtcatggttgaactgttgaatattgttcttgtactggtggaagaaactaacactcttcctgaatgaatgcaggttacaactggcttgcttatcgctgaagtcaacgtaaatacaatgtgcgagttgggttctggcggtgtctctttggtgatttattccttaacattatcttcaggattttttatattttgaatggacaagaaaaatcagctctcttactccctcagggaagcatgaattaggccaagagttggttacaaccatgtgattagcaagtttcgtttcacctcccttgtgattctgcataactttcaaatcattgtagtgatctaatcaattatcatgctttgttctttttagacttcgaacttagattttgtttggaatgtttgatgtttgtggtttataggaactctcatttgtcaatctcatttggttcaaagaaatgttccattttttgtctgtcagttcttaaagacctttttctttccattagaagtcagcagatccttgaacaagttaaatgtttgcttttccgaaacacgtaagtaaaaaatgcctgtaaatgaaccataggatctggtttgtctttgctacgagtcaagtcaggccttccatcatctatacctactcaaggagaaataagggcaattaactttttcttaagtttgttagaaagcttttttgggtaagaagttggttaccggaattcatgggattttgttatgcaaaagagtggtctagaagttttgtgaatgtaatctataaataggggaaagccattcagaagggggtatgttgatttgttgagtagaaactcttgatgggagaaacgcacccctcgaattgtgatacccattgtattgattaattttcttgattaattaatacaaatatccttagctttcctctaaatttctgctaactctgttattttcctgctaatatgatcactgtaatacataaacacaattaaaaacaaccaatcagacctggtatttagcagtctttctacggcgcttttttttgggttttctgaatgctgaatcatgcaatatgaacggttgcaacttaataatttgataagagagattgcatatctattctcccaatgcaattgtgatctgtacataagtttgcagcacaacccattgactgaggctattatgtttctgttctcactaccttttttttaaaagaaagatacaccaaaagttagggtagtcttctctcaagactttaccctaacttaacttttctcactacttgtcatgacctagtctataaaatctccgtttatactgtcggctttcagtaatttttttgaagaaagaatggttgcattcctcctttctaatgcagacatgtgacagttggatattgttttcaggtgtcaatggcaaagagaaccatagggtcagtgggagtccaagtcgcttggtaagacactttcttatgtattattatgtgcataagcagcttctattgtcttctatttctgaagatcttttaaaagagaaaaagaatttctacttgacttagtccaggctctaaacttgcgagaactcatttcagtaggaatgtttctgcttgagaatcctcgaacctcaaagttagtctgttttctgttgcttaaacattcagaatatgcatccaaatgatcttgtaaactttggataggtataaccagagtcttctctaatattgtggttctaccgagctctggaatgtgaggataggagtctatttatattactccttccgtccctaaaagattgccccaaatgcttttattcactttattaaaaattgggcatgctcaaagtaagtgggtagatagatttgtcttttagttaaattaaataatagtgtgtgaggagagacatgtgagcaccacaacccaaataaggtatgggtctaacaaatagggacatccatttatggtacacggggcaatcttttacggacagatggagtaccttgctatgcatagagttgtagatgtaagtctaggtgaaagattgggtgttaacaacgagtaacacttagacgataagagcttgtttgttggggtgtctgcctgtctggtttaaggaaaaaagtccagactgagtggagaagatatatatgtagaaaagaagacattagtgtgatatagatttgtatcattttccctcttcacaccattactttggatgctatgtctagcacggcattcaaattgagtcacattagctagacttgaaaaaactcaaaataatgtaactcaagtttacgagcaaaacatttcctcttttactttttgagcaatatggaagcaagaatgtttaagtatctattatgtctgcttcatgaatctattaaggattatttatatgttgattgttgactaacgactgtgttttccttgtactgcagctggtcatacatttttatacattacgctctccttgttgcctacatagcacgttcttcagatattttgacaaactaccttggtattccacagtatgacctatgcttctagcaattttctttcctttgaatttggtgaagtataagtggatgtcttctggtattttttatttcttgaatgctcgtagacaattttattttaatagtagagatcttgtcatccttgcgtgtatgagagtattgcagcttctagttcccctcggttttacactcttacataaatgattccgcccatgtttccatgtcctatacaaggtttgaattttgaaatgtcactacctgttttcttaaagcctgttttgtgcaaaatatttcaaaaacttgggtagtgtgtttcacggtttaccttataggaaattcctggataagaacatttcccttctttcaaaaattttggtagtgtgtttctctaaatgcatttcacaggcactcttgataagaaatttgttggctgattgctggggcatacaccttcattattctgcttcttcttcttacattaccgaattaccactagcaagtgtcatttgaaaactaagtaaatatcttgtgaataaacaatacagcaggtctttttatgaattattgcaatcataatcagtactcatgtaagtctagtatacaatttttgatggatttaatcgtttttgatagatgggagagtgcggcgttgttttcactggcttttggagcattatgttactttggaaggtgcgtttgagccttgtgctctattttattgcatcttgctcaatgtgaaagtatgggcatgctcgtttgtctactccatagcccttaacatctcctttttgcaaatatattaaagagagtgaaaaagtagcgagaaaagaaagtgagtggttggatatgggtccaactttagaagcatttttggacaaaaggaacttttttttgtcataatcgaccatgccttgtttttcatctcctttgtgccttgtgctttcttgttcattgatgatgctatttcattgtttagtaagatgatttgagaattgagaccactgttacctgattcgctagtatgtgactgtgactgtgtacgtgttcgcaattcgctagtacgtgttcattgatgatgcaattttcatgttataattcactttttgggttcactgttcgtgggatgattagggaattaactctatgtctctgatatttgataagcttagcaaattattgcatgagtttgtactttgtaatgcaaggatgaaattggtttctactttttatcttgcagccaacgatttataggtgccgttaatggactcttattattggggatcgtcacttcatttacagctctagtggtaagactttcctctttctgagctgaatatcccttctcccgtttgccttttctttctttatctatgaattttattcgggtttacagtacggcctctctcttgattctgtgaatagaagatgattttgttatctccaggtcattactagcgcagacctacattttgatgctcttttgaaggccaattttgaagccgtgccttcaagtttacctgtcatagcacttgcatatgtctaccacgtaagtaagatattaccttacatccttcaaagatacaaaatgtcttgtgcgcacaaggtgtacaataaatttattgtacaccaagataactttaaccctttttttttgtaactttaac

SEQ ID NO: 2 - DNA, 17496 bp, Spinacia tetrandra
  misc_feature 1..17496: note = second genomic fragment genomic DNA
  source 1..17496: mol_type = genomic DNA
gtttctacttgctcgtgaagtggaataatgtccaccacatgatttataaactgccaattgaaatagctattcacacctaaagatagtcgtgggccgggcagggccaaccctaaaagaaccggacccaacattggcatgacaaagcatggcccggcacaaacaaagttgtgggttgtgggttgttgcatttttttttttttggaaaaaacacgacaagacacggcacgataaagcatactaaatttagtaaaagcaggtttaggcacccggcacaactgccaatgggccggacctgggccatgttttgaacttttcagcccgtcctgccccagcacaaaggagaacgggcccagcctagcccggaccacggccttcttcttgcaccttgtcgactctgaaaccaaacatgttacagatagcaggctcacagagctaaagattgtttgcagaaataccgaattagtaaccctgatgaacaaaactggtcaaatatttgttaattacatgtaaagttgtaaaccatgataatttatgatggtaaaaggttaaatatatatgtgcagcttcttctactgacaaagcttgctatcaagaactctgctccaaacaccttattttcttcctgttttttccagcttttcacgaaattttcatgtgtggttgaaggtgttgagtatctctgataaaatgatccatgcagcgccacccatcactagcgaaagagtaagctttcccccgggaaccatctggggtactttcgggagttctggtgaagtcaagtatcgatctgaccacgacattgctgcaggaagaaccccgaacaacaccaaaactgcgagatttcagacactgttaaacagcttgacatatacgccctccatccctagccatgtttttattttaggtcgtcttagggctagcaatccctggcccgacccgataacctgactcaaacccgacctaaagttactgagagaaactgaacccggcccgaatgacctgaaatcgaccaaatttgacccatttatcatattttgtagtaatattaagttttactccgtataatatttctgatattttttgacacaacccaaaatcgacctgaaaccaaacctggacctggcccattgacgcgaattgccaccctaacaatatagaaaagtttctaaatttagttaattagatactcgacttctcccctaataccggcaatgatacccgtaactagaattccaaaggaagcaatgttatggtggagatgtaaaactatgcgtactggataactgcccgttcggtatcactttttgtttccagttttctgtttttttacaaaactaaaaatcaaaatatgaaaaccacaaaaagtaatttcccgttttttattgtcagttttcaaaatcaacatgattactataggtatgattattttttttgctcattattcaatctaattcatataactttcaaaaccaaaaaaaaagaaaaccggcagtgatactgaacttgctctcattgtcgctcgatttcttaaaaaacgaccctctaaaagaatctagggacggaaggagtagcaataatgaaattcgcaaaattggacaaccgaaagaagcaacacatgagcttaccgccatatgttccagcaaagtccaaagccttgaaaaagatctcagggtctaacaatgccagtaccaatggtgggaacaaagtaatcagatacggaagcggtttgctttgtcctgctggcagattcagcactgcaaacacggggttaggatgaatcagtcttcgattccaatacctcaaactgtatgtatggcagaaaattacaaatatagcatcatctacatacttaaaatgaagccaaaaccaataaaatttctacaatcttctcgaaaattatgtttaaggtggctaatcataagaataacgcaagtcttataaagattatgagcaggtaaaggagaaatgcttacagtctgctaagaagtcagcaagtccaagaacaaacccgatgtatgatgttccaatagcgaaaattgagaaggtatcgacgatgggctgcattacatcaataccaaaaacagaagcaatgtgtaaaattggtgaagctgcttcctcagtagacctgatcaaaagccgggctgggccgagacattaaattagcccatctgcccggttaggccaagatttgggccgatttttggtgcccactccgctatttcggccctaaatatcgggcttttggtcattcgggtctggtcaaatttataactaagaacgtaaatttatgttgcccaagtccatcaaaattttaaaatttcgagacggccgtgcccaaacccacaaaaatgcgggccgggctcatgttcaaaaaaaaaaaacatgagcccagcccgcatttttgtctgccttgttgatcaggtctattccacagtactcgaagtataaaagagctcaaaagaagagacttaccccgacaactccattgcttgctctcagctgttgtattggatcaagaatgttcttcccaacatccgaatctacatttgtgaatgaaccaagaattaccccgttccaaacaagaaatagaaccagaggaattgcggttcccatgatgactgcagtcctgcaaaagaacagagttagttcattcccagaaacaagaacttcagaaggtaaaatacaaaacagcgcgtggcaggatgtgagtatcacattatgcttcttatgtgaaaagcttgacaaagtaagttcacttcagtaccatgcaagaggattaagtgcacataatcataatcattatctgtcttcgatcaacaaaatgttacaatattagaatttgtgcaatagcttgatatgtactagtttacatgtactccctctgtctgaattaataatcacatatctgtagtaaggagtactattttacaggatatccaaaaaacggtttgtcacgtgcctaatatagcaatgccttatgatcatactacctccgttccttaatgttctttacacttactatttgcacagactccaatgcaatgtgaaaatattataaaagttagacattctgaaaatacataccatgtatataataagtgagaatttgcggtcaaagttttcatactttacacacatattccgaggagtaaagaactttcaggaacataggtagtattttctaatgagtcaatgataagaagatgatggaaatggccaaacggcccaactattatgaaaggtctttctgtaaataatgtcaaatcctgcattgtgaatggttggatcaacgtaacaactaattctggacaatgaaatactacaaagtatatgtatttctttgcagatcagtcaatatcggttctagtaattggtcaagaaaacacaacaatgcaactaaacacacatcacaaaattgtctatagataataatatgcagcagtgataagaatgctaatcagagtttcaccttacttttgacaagtcaccctccaagtttgtgcaaagaacaggaaccacgttctaaacagaccaaaaaaatagatgtgcgtcattaacaaaactaaagcggtaaatcatacggttagaactagacctgtcaaaaatcaacccgacccgtaaacccgacccgtgttttgggttttgagcgcgattttccaacccgaaacccaaataaatttggacaatgggtgtacaataaatattgtacaccgaagtaaaagttaactcaaaatgcttaaaagttaagcttatatatgtaaaagttgtctatttttaagtgataaattttttcattttagtaaaacttatttcttcaaaatcactaataatgtataaaattaatcatttaaccctttaaaatatttatctatcaattttttttactaatataaaagttaatcaaaactaggttaaagttacaaaaaaaaagggttaaagttatcttggtgtacaataaatttattgtacaccttgtgcgcacaagacattttgtatctttgaaggatgtaaggtaatatcttacttacgtggtagacatatgcaagtgctatgacaggtaaacttgaaggcacggcttcaaaattggccttcaaaagagcatcaaaatgtaggtctgcgctagtaatgacctggagataacaaaatcatcttctattcacagaatcaagagagaggccgtactgtaaacccgaataaaattcatagataaagaaagaaaaggcaaacgggagaagggatattcagctcagaaagaggaaagtcttaccactagagctgtaaatgaagtgacgatccccaataataagagtccattaacggcacctataaatcgttggctgcaagataaaaagtagaaaccaatttcatccttgcattacaaattacaaactcatgcaataatttgctaagcttatcaaatatcagagacatagagttaattccctaatcatcccacgaacagtgaacccaaaaagtgaattataacatgaaaattgcatcatcaatgaacacgtactagcgaattgcgaacacgtacacagtcacagtcacatactagcgaatcaggtaacagtggtctcaattctcaaatcatcttactaaacaatgaaatagcatcatcaatgaacaagaaagcacaaggcacaaaggagatgaaaaacaaggcatggtcgattatgacaaaaaaaagttccttttgtccaaaaatgcttctaaagttggacccatatccaaccactcactttcttttctcgctactttttcactctctttaatatatttgcaaaaaggagatgttaagggctatggagtagacaaacgagcatgcccatactttcacattgagcaagatgcaataaaatagagcacaaggctcaaacgcaccttccaaagtaacataatgctccaaaagccagtgaaaacaacgccgcactctcccatctatcaaaaacgattaaatccatcaaaaattgtatactagacttacatgagtactgattatgattgcaataattcataaaaagacctgctgtattgtttattcacaagatatttacttagttttcaaatgacacttgctagtggtaattcggtaatgtaagaagaagaagcagaataatgaaggtgtatgccccagcaatcagccaacaaatttcttatcaagagtgcctgtgaaatgcatttagagaaacacactaccaaaatttttgaaagaagggaaatgttcttatccaggaatttcctataaggtaaaccgtgaaacacactacccaagtttttgaaatattttgcacaaaacaggctttaagaaaacaggtagtgacatttcaaaattcaaaccttgtataggacatggaaacatgggcggaatcatttatgtaagagtgtaaaaccgaggggaactagaagctgcaatactctcatacacgcaaggatgacaagatctctactattaaaataaaattgtctacgagcattcaagaaataaaaaataccagaagacatccacttatacttcaccaaattcaaaggaaagaaaattgctagaagcataggtcatactgtggaataccaaggtagtttgtcaaaatatctgaagaacgtgctatgtaggcaacaaggagagcgtaatgtataaaaatgtatgaccagctgcagtacaaggaaaacacagtcgttagtcaacaatcaacatataaataatccttaatagattcatgaagcagacataatagatacttaaacattcttgcttccatattgctcaaaaagtaaaagaggaaatgttttgctcgtaaacttgagttacattattttgagttttttcaagtctagctaatgtgactcaatttgaatgccgtgctagacatagcatccaaagtaatggtgtgaagagggaaaatgatacaaatctatatcacactaatgtcttcttttctacatatatatcttctccactcagtctggacttttttccttaaaccagacaggcagacaccccaacaaacaagctcttatcgtctaagtgttactcgttgttaacacccaatctttcacctagacttacatctacaactctatgcatagcaaggtactccatctgtccgtaaaagattgccccgtgtaccataaatggatgtccctatttgttagacccataccttatttgggttgtggtgctcacatgtctctcctcacacactattatttaatttaactaaaagacaaatctatctacccacttactttgagcatgcccaatttttaataaagtgaataaaagcatttggggcaatcttttagggacggaaggagtaatataaatagactcctatcctcacattccagagctcggtagaaccacaatattagagaagactctggttatacctatccaaagtttacaagatcatttggatgcatattctgaatgtttaagcaacagaaaacagactaactttgaggttcgaggattctcaagcagaaacattcctactgaaatgagttctcgcaagtttagagcctggactaagtcaagtagaaattctttttctcttttaaaagatcttcagaaatagaagacaatagaagctgcttatgcacataataatacataagaaagtgtcttaccaagcgacttggactcccactgaccctatggttctctttgccattgacacctgaaaacaatatccaactgtcacatgtctgcattagaaaggaggaatgcaaccattctttcttcaaaaaaattactgaaagccgacagtataaacggagattttatagactaggtcatgacaagtagtgagaaaagttaagttagggtaaagtcttgagagaagactaccctaacttttggtgtatctttcttttaaaaaaaaggtagtgagaacagaaacataatagcctcagtcaatgggttgtgctgcaaacttatgtacagatcacaattgcattgggagaatagatatgcaatctctcttatcaaattattaagttgcaaccgttcatattgcatgattcagcattcagaaaacccaaaaaaaagcgtcgtagaaagactgctaaataccaggtctgattggttgtttttaattgtgtttatgtattacagtgatcatattagcaggaaaataacagagttagcagaaatttagaggaaagctaaggatatttgtattaattaatcaagaaaattaatcaatacaatgggtatcacaattcgaggggtgcgtttctcccatcaagagtttctactcaacaaatcaacatacccccttctgaatggctttcccctatttatagattacattcacaaaacttctagaccactcttttgcataacaaaatcccatgaattccggtaaccaacttcttacccaaaaaagctttctaacaaacttaagaaaaagttaattgcccttatttctccttgagtaggtatagatgatggaaggcctgacttgactcgtagcaaagacaaaccagatcctatggttcatttacaggcattttttacttacgtgtttcggaaaagcaaacatttaacttgttcaaggatctgctgacttctaatggaaagaaaaaggtctttaagaactgacagacaaaaaatggaacatttctttgaaccaaatgagattgacaaatgagagttcctataaaccacaaacatcaaacattccaaacaaaatctaagttcgaagtctaaaaagaacaaagcatgataattgattagatcactacaatgatttgaaagttatgcagaatcacaagggaggtgaaacgaaacttgctaatcacatggttgtaaccaactcttggcctaattcatgcttccctgagggagtaagagagctgatttttcttgtccattcaaaatataaaaaatcctgaagataatgttaaggaataaatcaccaaagagacaccgccagaacccaactcgcacattgtatttacgttgacttcagcgataagcaagccagttgtaacctgcattcattcaggaagagtgttagtttcttccaccagtacaagaacaatattcaacagttcaaccatgacaatcaaatacatgaatttgctgtaaaagaggcagaaagaaactatatagctatggagaataaatattagaacaccttagtgccaagcccttaccatatatacccaacaaccaatgcacgcgacagcagaggcaagaaacccagattcttgtgtcacagcaggaagtgctaaaatacctgcacctatctgtaaaattgtcacagaaaatgagtcataactgaacagaaaggaaaaacatagtgaaaaatggataataaactaatcatatcagggaacatgtagtagagcaagcaatatatacaccatacaaaaagaacaaagtagaacatgtaaaagcaatatagtatatcgcgaattttgcaaccaaaatgccattttcaaaatgcttccagcagtcagatgcttcattgtaatgttgaagttccaaataatcatcattcatcaccatagaattagacaatcacacttgcattaattacaggaccggacattttccctaggcatggaatgtactaacattgcctctttcacttgcattaatcccataactatgaggtaaaacttgaacatgtaaaagcaatatagtataccgcgatcaaaatgccattttcaaaaactgtaaaagctccattaatcccctaaatctgaacatgtaattgcaatacagataccgtgtccacgatcaacattcgtgctttcactagcattaaaacacacacacacacacacaactcacacacactcacacacacaactcacacacacacaactaagaggtgtgcactaataactaatccaacattcttaattttcactaaaaaaagctcaaatttcattcaataagctaaaattggcaactatagtctatagcataccgtagtgcctgccacaaggaaaattgaactggtaacactacctgccaacagggtaaaatgaaaaggaaataatcaagaaaacaatcaaacttcaaaataatgaaaatccaatctataactaagcaatatgagaaaaattaagctaaaattcaaaccaggttcatgcttcaaagtggcttgattaatgtttgaaaacaacctctcaaagtcatacttttgaacttcttttcctacttcttcttcctctttctctacttgattgagtgaattttgggtgggttggctttgtttttgagaaaaacatcttaagctgggttttctgaggtgattagacaaatgggtattgggaaattggggaaattgaacaggaattaagggtttaatttgatgaaaatgatggtggtgacagaaaatttggtgggttttggggttagagaggggggtgccaatcaacgccatttttctgtcatcaagttaaaaaaattcccagaaattaagttggttaattcatgagttactggaatgggccacgttatctgcaagaagattcattttagttttgtggttttgatcagtaggatgacaattgacaacttcgggtttttttcggttcatctttttttttttctttttttttttttttaagcatgaacctggttttttaatcaagccacaaatatcaaacattccaaacaaaatctccctttatttgtatgtctcatattttttatatttttttaccatttttaacgctaatttggattcggggtgagttttggagtagatttgttattgtaggggatcgaacacacggttcgccctaccaagttcaaccccagtcatcactgaaccaacatgtaattgatatgtctcatattagtcgatagtctcacaccactagctagtcactagactcactacaatactttttacagttcttaattcttacatgatacggagtaaaattcattaagtactatcccattcacatagtatgagactcaagacaagttatatacgtagtaacaatctaattaatcataatattgacacattttaacaaaacattttgattttggcggaaattgatgtaacttaataaaaagctcgatagttttccattaaaaaaatgatatatttttctactactcgggtatcgacaggaccacttatccatccaaaatgatgaacggaatagcacgggattacagtttctattggagaaaaaggaataatataatccctccatattttttttaggaattatatgttagccggcacaaagattaatgaagatgagttaattaacttacaataatgataccaatattgcatgtgtaattatttattgtaaatatctgatatggtctgcccagattagggatgtcaatggggcggggcagatgtggatgtaagtttcccatccccatcccacctcaaattttcatcccatctccgtcccatcctccatggcggatacaaaatttatccccatctcttcccccaacaggtacaaactaaaatccatccccgccctaccacccgcttggttatccatcccgatttcatcccggcccaaccatacccgtgccaatactgctatttttttttttttttttttttcttatttagcaaacatttgacaacatatataatgaattacggagtaacttttaaaaataccttacaactaaaactaacatatacgaattataatgaaaaaaaaacacattaattttaattgtagattttctttaaaagttttaattcacgtaggaaaaaataccggatcggattgacatacataaagtggacgggtctaacacaaaatccgcaacgcgccatcactcatagtgggcacaatttttatatgcattcccactcatcactcgtcaaacctccctgtccccacctacttggggcggatgcgaggcgggtcgggtctcccattaaacccgctccacttacgtgcctatcccggtgacccggatattgctcattacggagtacggaataacacgaaaaccttttttttttactccgtatctcttttgcgcgcaatgtactctgattattttaaaaaaaaaccaacccaaatctggacaaaaacttaaaccatgcaaaattaatccactttgaacacaaacttgaagagaaccaaaaaaaataaaaatggagcgaaatatgaatagtgaagtcgcgagaagttcttcaatttcaagcttatcaagcgacaacaatggcgataacaagtctatcgtcgttagggttaagcgcaagcttgaccaatctcctctcgacgctttctgtatgttcctcttctattttaattttcttttaaatttctgcaaaattaaatttcacttttgatgatttttgcaatgcaagggttgaaatcaacgagagatctgttgtgcgtctgttaatagattttgatcagctatccgtttcgatctgattcttctactcaaggtatattctaattttggttttgatagttgtaattctgggtttattaaagtataattacttaattagggtgattatgatgtgattagtcgactaattgggcggaaaacgtattaattgacattgagttagtaattgacctcctttgttagaaaaatagctaataattgacaacttttaacaaaacctatgtagtaattagttagtaattgacaacttatcagtgaagagtacggagtagttagtagtaattgataaagttctaaacaacctatgtcgtgcttgacaaattttccactacacagcaatggaaatgaattgtacatttctactctattgtttttgtgcgttgggcccgaacattaggtcatgtcttctctttcaaagacttggttatttcgacttgactccggtggggctgtgttgagtatggttatacagttgttttgcttgacctgatcctaaacttggacctaactctgtattacttgaacgtttgcgtacgccctctgtccctcgtagacaatgtgtgcttaattaagggttgaaccgaaaatttacaaggaatttgggggtatctatgtctttttgactttttgagtgtatacacgtgggataaatggtggggacatgttactatataaggaaattagaaaccggacaatttttaaaggatggaccaaaataaaatatggggaaaacaaatagggacagagggtatcgaattatcaattatcctccacaaaagttgaggaaaatcaacgtcgttgtcaagttgggcctgagatgagatgggtggaagaaaatgagccctttcagtatagttgatgcagcaagtaggaaagttgcaatatgtcaagtcaactcaagtaaacgaagaaaaatgtggcttaagcaagtttgtcatatgttatttacttgtgggtctaccaactaaccttgaaaatcacttgtactgttgtgcatactttggacaagacgatgtctttgcttttctcactctttcctcactctttcctcgaattgtaagacgttcttccaacccaaacacaatacaagtatatgcctttatttataacggagtagaggaattttaaaactcacttcattatttcctttgcattcctcaaaataagcaatactaattcataacaaaagagagagagatggatgtgtctgcaggcctttcactagtacaaactgtcctcgaattgttgggttctcccatttggacacagattcaatctttgttgggtgtggagtactcacagcttgacaaactcaaggccaccatgtccactattgaggctgtgctccttgatgctgaagatcaagagcagttgcatcaatatcgtcgtagccatgttgaactggataggcttcagaggcttaaggaagctctttatatagctgatgacttgttcgacaagattgccactcttgctcaacgcaaaaccctcataatgcgctttaataacaagctgcttaatgaggtatgccttttcttctcttcctctaataagcttcgttctgctttcactgggtctcgtgagatggagaacattagggagatgttgaatgatattgtcaaagatcatcgctatgactttggctccagacaccccgttagtctggatgcgaaacagctgagaatcaaagcaagggaaactcattcctttgtttgcgaggaagatatgatcattgggagggatgatgacaagcggaaagtgatagatatggtactaaacacttcttgtgaggagaaggatatttctgttgtcagcatagtggggattggggggctgggaaaaacaactctagcccgacttgtttataatgatgagaagataggtagtaaattctctctgaaaatgtgggtttgtgtctctgatagtttcaatgtcaaagatcttgttgctgaaatccttgcagccgtaaaccctagaagaaagcacgatgccctgagtatgagtatggaccagcttcagatgaaaattcgggacgaactggatgggaagatatacctccttgttttggatgatgtatggactgaagatcccattaaatggcatgagctgagaaaactattgattggaggtagaaacggtagcagaattctggtgactactcgctcaagtggggtggcgaaagttgttggaagctatcatattcataaattggaaggactctctaataaagagtcttgggatttgtttgagaggatgacacttgaaccaggacaacatcaaatgcaagagcacttggttaaaattggaaaagatattgtgagaaagtgtgcaaatgttccacttgctataagggtggttggaagtcttcttcgaggtcagggtgaaagtaggtggcaatatttgaaaaacactgacttggcaaatataccacaagatgagatcaatggtattttacctgttttgaagattagttattattatcttccgttccatttgaagagttgctttaagtattgttctgtattccccaaggattacaagattatcaaggaggatttgatcagtctctggatggcacaaggcttcatcatctcatcaaatgaagagagtttcgaaaatgcaggtgaggaatatttcaaacaattactccaaaggtgtttcttccaagatgtagaacgggccgagggtactgatgaaattctatcatgcaagatgcatgacttgattcatgaccttgctagtgaagtagcaggaacagaaatcgtgtcgtccaagtataatataagggggtttagcgagaaaactcgccatatatttattgatagagatgccgttaaagatatttatcgtcatttcaccaatatgaaaaggatgcgttcaatttttagaatagatgcgctttcttctaattcaacatatttgttatcaaaagtggaatatttaagggtcttaagcttgaataactctgatttggaaatgttgccgagtgagataggtaacttgttgcatctaagatatcttgacctatcagataattaccgtctctctaagttgcctacctccattaccaagctttacaacttgcaaatattgaagttacgtgagtgtcgtggtctaaaaactttgccaagcgatttgaggaaattagtaaatctaaggcacctagatatacaaggctgtaagagtttgtcgcatatgccccgggggatgaatagcatgacatctctgcataaactgacagggtttgtagtgagcagaaaaagaaatagctggaatcgggggtcagttagtgttggtgagctgggagatttgaaaaatttcagcaatcatagccacaatatggtaatttatgtcaaaaaagatgcagttgatgctagggaaaaggggtttctattaaaaagtgaacatttgagggaaattcagtatgtttggaatcatgagagtgaggggactgatgcagatgcattgctgcaagggaaaaatgcagatgtggtgctgcaagggctgcaaccccatcttaatctccgaatgttggagttgagggattatccaggaattaggttcccaagctgggggaggtcatcaatgaatctccatacttgtctcccaaatcttgttagcataacactatacggatgtacaaggttggagcatcttccattgatgagtcaactgcgtcatttgaaattccttacattacgatatttgagtgaagtggtgtatatggagaatagtattatcagcgcagaaggggttgcatcgacatcaggagggtcaagtcatggaggagcagatgaggacttggttttctttccatcccttgaaaagcttgagctttggagtatgccaaagttggaaggatggtggaaattagaatcagatatgggtgagacacgagaagtagcagggtttcaaacacattcatattcgttccatcacctttctcatctgttaattacattttgtgataatttgagaaattttcctctctgtccgaaactggaagaatcaaactcccaactacaggaaatcagaaatctgaatcattcttttaccagtaagatcttcttttttattgtgtgtgtgtaaaaggttacatatttttatatatacctagtagctcctaactatctacaagtataactttgttcatggtataagtgcagaagacgaagttgagtccctccaatccttgcaatttgaccttgaaactatagaaattgcaacacataacttttctgatgataataagatcggagaaggcatcggcccggtttataaggtactaattcagttaatatagtactggtgtactacgtattagtgtattacttcaaatgattaaccagcttatttgttctttgtagggtatacttcctgatgggcaagagatagaagttaagaggcttttaaggaactcgcgccaaagagatgcagagttcaaaaatgaaattttgatattagccaaggtccggcacaagaatttggtgaaacttctaggattttgcttgcatggagaagaaaggattcttgtctatgagtttgttgccaacagaagccttgacaactttatatttggtatgtaatcactactacaattcgagattttacacatactaccgggatgtataacaatccaatccgggtgattttcaaatgattatgatgtatattagatttgtttacgactctaaggactagtttcattgcatgatacatgtatccagtgtccatcatactattttggtaataaaatgcagatccattgagtcgtcagtctatgcaatgggaaattcgttataagattatcaatggcatagctagagggattctatacctccatgaaggttccagactaatgatcatacatcgcaacctagaagcaggaagtgttctacttgatgaagatttttgtcccaagattgcagattttggtatggcatggctatttaacatagatcaaactcaaacagaggctagcagaatcgctggaaccttgtaagcatcggaactttatatttagttaattacataatgtctaatactctgtatgcttttgttttcttattttgagaaacgatttttgttattcagaggatatattgcacctgagtatgctacacaatatcaagtatcagtaaagtcagacgtatacagctttggtatactagtcctagagatagtaagcgggcagaggattacttcatttcattacgagggagaaaacctcacgacctctgtaagtttgattatctgattggttaattctaaatttacgttgcaaaaaattgacaaacttgacctcattcatggatgacatttgtgtataggcatggagaaactggaatgaaggcacagcttggaatctggtagatcctgtgttactaggcagctctagaaccgagatactgcgatgcatccatatcggtttgttgtgtgtccaagaaagccctgcagacaggccaactatgtctgaggttcatgttatgttgagtacgaattcggtcacccttgaagcacctttgcagcctgcattttgtacagatagccaaagtctacccgatatgcttcgggagtggagcacaactaatagtcaagtatcagatgatgaatcggagccgtaggggaggtgataaatccaaggaaaatttacttcttccatggaaatccacatttttcaaaactaattttcttggaaaaagtgatttttccccccttggatttgtcatttgccaaacaggagtagttcagtaaatggtgtttatgtaacagagttgcaccctagatcatgtaattttttagatttcaattggtgtatactgtataggggcttgattgatataatttctaggtcattttgtgtagcaaagtgataaaggtcgtaacttgcgacaaattatttgtgttgtaaatattgacgtgtcacatattaaaattgggaattagagattttgtaacataaacacactattagtagactaaattatatagcagaacttgaatttcctcatttgcaatatgtaagttttcttttgaaactatatacttcctccgtcttttaatactagcaacgttttgactttttccactatttatataatataattttacta

SEQ ID NO: 3 - DNA, 3693 bp, Spinacia tetrandra
  misc_feature 1..3693: note = frirst cDNA sequence
  source 1..3693: mol_type = other DNA
atggatgtgtctgcaggcctttcactagtacaaactgtcctcgaattgttgggttctcccatttggacacagattcaatctttgttgggtgtggagtactcacagcttgacaaactcaaggccaccatgtccactattgaggctgtgctccttgatgctgaagatcaagagcagttgcatcaatatcgtcgtagccatgttgaactggataggcttcagaggcttaaggaagctctttatatagctgatgacttgttcgacaagattgccactcttgctcaacgcaaaaccctcataatgcgctttaataacaagctgcttaatgaggtatgccttttcttctcttcctctaataagcttcgttctgctttcactgggtctcgtgagatggagaacattagggagatgttgaatgatattgtcaaagatcatcgctatgactttggctccagacaccccgttagtctggatgcgaaacagctgagaatcaaagcaagggaaactcattcctttgtttgcgaggaagatatgatcattgggagggatgatgacaagcggaaagtgatagatatggtactaaacacttcttgtgaggagaaggatatttctgttgtcagcatagtggggattggggggctgggaaaaacaactctagcccgacttgtttataatgatgagaagataggtagtaaattctctctgaaaatgtgggtttgtgtctctgatagtttcaatgtcaaagatcttgttgctgaaatccttgcagccgtaaaccctagaagaaagcacgatgccctgagtatgagtatggaccagcttcagatgaaaattcgggacgaactggatgggaagatatacctccttgttttggatgatgtatggactgaagatcccattaaatggcatgagctgagaaaactattgattggaggtagaaacggtagcagaattctggtgactactcgctcaagtggggtggcgaaagttgttggaagctatcatattcataaattggaaggactctctaataaagagtcttgggatttgtttgagaggatgacacttgaaccaggacaacatcaaatgcaagagcacttggttaaaattggaaaagatattgtgagaaagtgtgcaaatgttccacttgctataagggtggttggaagtcttcttcgaggtcagggtgaaagtaggtggcaatatttgaaaaacactgacttggcaaatataccacaagatgagatcaatggtattttacctgttttgaagattagttattattatcttccgttccatttgaagagttgctttaagtattgttctgtattccccaaggattacaagattatcaaggaggatttgatcagtctctggatggcacaaggcttcatcatctcatcaaatgaagagagtttcgaaaatgcaggtgaggaatatttcaaacaattactccaaaggtgtttcttccaagatgtagaacgggccgagggtactgatgaaattctatcatgcaagatgcatgacttgattcatgaccttgctagtgaagtagcaggaacagaaatcgtgtcgtccaagtataatataagggggtttagcgagaaaactcgccatatatttattgatagagatgccgttaaagatatttatcgtcatttcaccaatatgaaaaggatgcgttcaatttttagaatagatgcgctttcttctaattcaacatatttgttatcaaaagtggaatatttaagggtcttaagcttgaataactctgatttggaaatgttgccgagtgagataggtaacttgttgcatctaagatatcttgacctatcagataattaccgtctctctaagttgcctacctccattaccaagctttacaacttgcaaatattgaagttacgtgagtgtcgtggtctaaaaactttgccaagcgatttgaggaaattagtaaatctaaggcacctagatatacaaggctgtaagagtttgtcgcatatgccccgggggatgaatagcatgacatctctgcataaactgacagggtttgtagtgagcagaaaaagaaatagctggaatcgggggtcagttagtgttggtgagctgggagatttgaaaaatttcagcaatcatagccacaatatggtaatttatgtcaaaaaagatgcagttgatgctagggaaaaggggtttctattaaaaagtgaacatttgagggaaattcagtatgtttggaatcatgagagtgaggggactgatgcagatgcattgctgcaagggaaaaatgcagatgtggtgctgcaagggctgcaaccccatcttaatctccgaatgttggagttgagggattatccaggaattaggttcccaagctgggggaggtcatcaatgaatctccatacttgtctcccaaatcttgttagcataacactatacggatgtacaaggttggagcatcttccattgatgagtcaactgcgtcatttgaaattccttacattacgatatttgagtgaagtggtgtatatggagaatagtattatcagcgcagaaggggttgcatcgacatcaggagggtcaagtcatggaggagcagatgaggacttggttttctttccatcccttgaaaagcttgagctttggagtatgccaaagttggaaggatggtggaaattagaatcagatatggaagacgaagttgagtccctccaatccttgcaatttgaccttgaaactatagaaattgcaacacataacttttctgatgataataagatcggagaaggcatcggcccggtttataagggtatacttcctgatgggcaagagatagaagttaagaggcttttaaggaactcgcgccaaagagatgcagagttcaaaaatgaaattttgatattagccaaggtccggcacaagaatttggtgaaacttctaggattttgcttgcatggagaagaaaggattcttgtctatgagtttgttgccaacagaagccttgacaactttatatttgatccattgagtcgtcagtctatgcaatgggaaattcgttataagattatcaatggcatagctagagggattctatacctccatgaaggttccagactaatgatcatacatcgcaacctagaagcaggaagtgttctacttgatgaagatttttgtcccaagattgcagattttggtatggcatggctatttaacatagatcaaactcaaacaaaggctagcagaatcgctggaaccttaggatatattgcacctgagtatgctacacaatatcaagtatcagtaaagtcagacgtatacagctttggtatactagtcctagagatagtaagcgggcagaggattacttcatttcattacgagggagaaaacctcacgacctctgcatggagaaactggaatgaaggcacagcttggaatctggtagatcctgtgttactaggcagctctagaaccgagatactgcgatgcatccatatcggtttgttgtgtgtccaagaaagccctgcagacaggccaactatgtctgaggttcatgttatgttgagtacgaattcggtcacccttgaagcacctttgcagcctgcattttgtacagatagccaaagtctacccgatatgcttcgggagtggagcacaactaatagtcaagtatcagatgatgaatcggagccgtag

SEQ ID NO: 4 - DNA, 3693 bp, Spinacia tetrandra
  misc_feature 1..3693: note = second cDNA sequence
  source 1..3693: mol_type = other DNA
atggatgtgtctgcaggcctttcactagtacaaactgtcctcgaattgttgggttctcccatttggacacagattcaatctttgttgggtgtggagtactcacagcttgacaaactcaaggccaccatgtccactattgaggctgtgctccttgatgctgaagatcaagagcagttgcatcaatatcgtcgtagccatgttgaactggataggcttcagaggcttaaggaagctctttatatagctgatgacttgttcgacaagattgccactcttgctcaacgcaaaaccctcataatgcgctttaataacaagctgcttaatgaggtatgccttttcttctcttcctctaataagcttcgttctgctttcactgggtctcgtgagatggagaacattagggagatgttgaatgatattgtcaaagatcatcgctatgactttggctccagacaccccgttagtctggatgcgaaacagctgagaatcaaagcaagggaaactcattcctttgtttgcgaggaagatatgatcattgggagggatgatgacaagcggaaagtgatagatatggtactaaacacttcttgtgaggagaaggatatttctgttgtcagcatagtggggattggggggctgggaaaaacaactctagcccgacttgtttataatgatgagaagataggtagtaaattctctctgaaaatgtgggtttgtgtctctgatagtttcaatgtcaaagatcttgttgctgaaatccttgcagccgtaaaccctagaagaaagcacgatgccctgagtatgagtatggaccagcttcagatgaaaattcgggacgaactggatgggaagatatacctccttgttttggatgatgtatggactgaagatcccattaaatggcatgagctgagaaaactattgattggaggtagaaacggtagcagaattctggtgactactcgctcaagtggggtggcgaaagttgttggaagctatcatattcataaattggaaggactctctaataaagagtcttgggatttgtttgagaggatgacacttgaaccaggacaacatcaaatgcaagagcacttggttaaaattggaaaagatattgtgagaaagtgtgcaaatgttccacttgctataagggtggttggaagtcttcttcgaggtcagggtgaaagtaggtggcaatatttgaaaaacactgacttggcaaatataccacaagatgagatcaatggtattttacctgttttgaagattagttattattatcttccgttccatttgaagagttgctttaagtattgttctgtattccccaaggattacaagattatcaaggaggatttgatcagtctctggatggcacaaggcttcatcatctcatcaaatgaagagagtttcgaaaatgcaggtgaggaatatttcaaacaattactccaaaggtgtttcttccaagatgtagaacgggccgagggtactgatgaaattctatcatgcaagatgcatgacttgattcatgaccttgctagtgaagtagcaggaacagaaatcgtgtcgtccaagtataatataagggggtttagcgagaaaactcgccatatatttattgatagagatgccgttaaagatatttatcgtcatttcaccaatatgaaaaggatgcgttcaatttttagaatagatgcgctttcttctaattcaacatatttgttatcaaaagtggaatatttaagggtcttaagcttgaataactctgatttggaaatgttgccgagtgagataggtaacttgttgcatctaagatatcttgacctatcagataattaccgtctctctaagttgcctacctccattaccaagctttacaacttgcaaatattgaagttacgtgagtgtcgtggtctaaaaactttgccaagcgatttgaggaaattagtaaatctaaggcacctagatatacaaggctgtaagagtttgtcgcatatgccccgggggatgaatagcatgacatctctgcataaactgacagggtttgtagtgagcagaaaaagaaatagctggaatcgggggtcagttagtgttggtgagctgggagatttgaaaaatttcagcaatcatagccacaatatggtaatttatgtcaaaaaagatgcagttgatgctagggaaaaggggtttctattaaaaagtgaacatttgagggaaattcagtatgtttggaatcatgagagtgaggggactgatgcagatgcattgctgcaagggaaaaatgcagatgtggtgctgcaagggctgcaaccccatcttaatctccgaatgttggagttgagggattatccaggaattaggttcccaagctgggggaggtcatcaatgaatctccatacttgtctcccaaatcttgttagcataacactatacggatgtacaaggttggagcatcttccattgatgagtcaactgcgtcatttgaaattccttacattacgatatttgagtgaagtggtgtatatggagaatagtattatcagcgcagaaggggttgcatcgacatcaggagggtcaagtcatggaggagcagatgaggacttggttttctttccatcccttgaaaagcttgagctttggagtatgccaaagttggaaggatggtggaaattagaatcagatatggaagacgaagttgagtccctccaatccttgcaatttgaccttgaaactatagaaattgcaacacataacttttctgatgataataagatcggagaaggcatcggcccggtttataagggtatacttcctgatgggcaagagatagaagttaagaggcttttaaggaactcgcgccaaagagatgcagagttcaaaaatgaaattttgatattagccaaggtccggcacaagaatttggtgaaacttctaggattttgcttgcatggagaagaaaggattcttgtctatgagtttgttgccaacagaagccttgacaactttatatttgatccattgagtcgtcagtctatgcaatgggaaattcgttataagattatcaatggcatagctagagggattctatacctccatgaaggttccagactaatgatcatacatcgcaacctagaagcaggaagtgttctacttgatgaagatttttgtcccaagattgcagattttggtatggcatggctatttaacatagatcaaactcaaacagaggctagcagaatcgctggaaccttaggatatattgcacctgagtatgctacacaatatcaagtatcagtaaagtcagacgtatacagctttggtatactagtcctagagatagtaagcgggcagaggattacttcatttcattacgagggagaaaacctcacgacctctgcatggagaaactggaatgaaggcacagcttggaatctggtagatcctgtgttactaggcagctctagaaccgagatactgcgatgcatccatatcggtttgttgtgtgtccaagaaagccctgcagacaggccaactatgtctgaggttcatgttatgttgagtacgaattcggtcacccttgaagcacctttgcagcctgcattttgtacagatagccaaagtctacccgatatgcttcgggagtggagcacaactaatagtcaagtatcagatgatgaatcggagccgtag

SEQ ID NO: 5 - protein, 1230 aa, Spinacia tetrandra
  REGION 1..1230: note = first protein sequence
MDVSAGLSLVQTVLELLGSPIWTQIQSLLGVEYSQLDKLKATMSTIEAVLLDAEDQEQLHQYRRSHVELDRLQRLKEALYIADDLFDKIATLAQRKTLIMRFNNKLLNEVCLFFSSSNKLRSAFTGSREMENIREMLNDIVKDHRYDFGSRHPVSLDAKQLRIKARETHSFVCEEDMIIGRDDDKRKVIDMVLNTSCEEKDISVVSIVGIGGLGKTTLARLVYNDEKIGSKFSLKMWVCVSDSFNVKDLVAEILAAVNPRRKHDALSMSMDQLQMKIRDELDGKIYLLVLDDVWTEDPIKWHELRKLLIGGRNGSRILVTTRSSGVAKVVGSYHIHKLEGLSNKESWDLFERMTLEPGQHQMQEHLVKIGKDIVRKCANVPLAIRVVGSLLRGQGESRWQYLKNTDLANIPQDEINGILPVLKISYYYLPFHLKSCFKYCSVFPKDYKIIKEDLISLWMAQGFIISSNEESFENAGEEYFKQLLQRCFFQDVERAEGTDEILSCKMHDLIHDLASEVAGTEIVSSKYNIRGFSEKTRHIFIDRDAVKDIYRHFTNMKRMRSIFRIDALSSNSTYLLSKVEYLRVLSLNNSDLEMLPSEIGNLLHLRYLDLSDNYRLSKLPTSITKLYNLQILKLRECRGLKTLPSDLRKLVNLRHLDIQGCKSLSHMPRGMNSMTSLHKLTGFVVSRKRNSWNRGSVSVGELGDLKNFSNHSHNMVIYVKKDAVDAREKGFLLKSEHLREIQYVWNHESEGTDADALLQGKNADVVLQGLQPHLNLRMLELRDYPGIRFPSWGRSSMNLHTCLPNLVSITLYGCTRLEHLPLMSQLRHLKFLTLRYLSEVVYMENSIISAEGVASTSGGSSHGGADEDLVFFPSLEKLELWSMPKLEGWWKLESDMEDEVESLQSLQFDLETIEIATHNFSDDNKIGEGIGPVYKGILPDGQEIEVKRLLRNSRQRDAEFKNEILILAKVRHKNLVKLLGFCLHGEERILVYEFVANRSLDNFIFDPLSRQSMQWEIRYKIINGIARGILYLHEGSRLMIIHRNLEAGSVLLDEDFCPKIADFGMAWLFNIDQTQTKASRIAGTLGYIAPEYATQYQVSVKSDVYSFGILVLEIVSGQRITSFHYEGENLTTSAWRNWNEGTAWNLVDPVLLGSSRTEILRCIHIGLLCVQESPADRPTMSEVHVMLSTNSVTLEAPLQPAFCTDSQSLPDMLREWSTTNSQVSDDESEP

SEQ ID NO: 6 - protein, 1230 aa, Spinacia tetrandra
  REGION 1..1230: note = second protein sequence
MDVSAGLSLVQTVLELLGSPIWTQIQSLLGVEYSQLDKLKATMSTIEAVLLDAEDQEQLHQYRRSHVELDRLQRLKEALYIADDLFDKIATLAQRKTLIMRFNNKLLNEVCLFFSSSNKLRSAFTGSREMENIREMLNDIVKDHRYDFGSRHPVSLDAKQLRIKARETHSFVCEEDMIIGRDDDKRKVIDMVLNTSCEEKDISVVSIVGIGGLGKTTLARLVYNDEKIGSKFSLKMWVCVSDSFNVKDLVAEILAAVNPRRKHDALSMSMDQLQMKIRDELDGKIYLLVLDDVWTEDPIKWHELRKLLIGGRNGSRILVTTRSSGVAKVVGSYHIHKLEGLSNKESWDLFERMTLEPGQHQMQEHLVKIGKDIVRKCANVPLAIRVVGSLLRGQGESRWQYLKNTDLANIPQDEINGILPVLKISYYYLPFHLKSCFKYCSVFPKDYKIIKEDLISLWMAQGFIISSNEESFENAGEEYFKQLLQRCFFQDVERAEGTDEILSCKMHDLIHDLASEVAGTEIVSSKYNIRGFSEKTRHIFIDRDAVKDIYRHFTNMKRMRSIFRIDALSSNSTYLLSKVEYLRVLSLNNSDLEMLPSEIGNLLHLRYLDLSDNYRLSKLPTSITKLYNLQILKLRECRGLKTLPSDLRKLVNLRHLDIQGCKSLSHMPRGMNSMTSLHKLTGFVVSRKRNSWNRGSVSVGELGDLKNFSNHSHNMVIYVKKDAVDAREKGFLLKSEHLREIQYVWNHESEGTDADALLQGKNADVVLQGLQPHLNLRMLELRDYPGIRFPSWGRSSMNLHTCLPNLVSITLYGCTRLEHLPLMSQLRHLKFLTLRYLSEVVYMENSIISAEGVASTSGGSSHGGADEDLVFFPSLEKLELWSMPKLEGWWKLESDMEDEVESLQSLQFDLETIEIATHNFSDDNKIGEGIGPVYKGILPDGQEIEVKRLLRNSRQRDAEFKNEILILAKVRHKNLVKLLGFCLHGEERILVYEFVANRSLDNFIFDPLSRQSMQWEIRYKIINGIARGILYLHEGSRLMIIHRNLEAGSVLLDEDFCPKIADFGMAWLFNIDQTQTEASRIAGTLGYIAPEYATQYQVSVKSDVYSFGILVLEIVSGQRITSFHYEGENLTTSAWRNWNEGTAWNLVDPVLLGSSRTEILRCIHIGLLCVQESPADRPTMSEVHVMLSTNSVTLEAPLQPAFCTDSQSLPDMLREWSTTNSQVSDDESEP

SEQ ID NO: 7 - DNA, 999 bp, Spinacia tetrandra
  misc_feature 1..999: note = cDNA LRR domain
  source 1..999: mol_type = other DNA
agaatagatgcgctttcttctaattcaacatatttgttatcaaaagtggaatatttaagggtcttaagcttgaataactctgatttggaaatgttgccgagtgagataggtaacttgttgcatctaagatatcttgacctatcagataattaccgtctctctaagttgcctacctccattaccaagctttacaacttgcaaatattgaagttacgtgagtgtcgtggtctaaaaactttgccaagcgatttgaggaaattagtaaatctaaggcacctagatatacaaggctgtaagagtttgtcgcatatgccccgggggatgaatagcatgacatctctgcataaactgacagggtttgtagtgagcagaaaaagaaatagctggaatcgggggtcagttagtgttggtgagctgggagatttgaaaaatttcagcaatcatagccacaatatggtaatttatgtcaaaaaagatgcagttgatgctagggaaaaggggtttctattaaaaagtgaacatttgagggaaattcagtatgtttggaatcatgagagtgaggggactgatgcagatgcattgctgcaagggaaaaatgcagatgtggtgctgcaagggctgcaaccccatcttaatctccgaatgttggagttgagggattatccaggaattaggttcccaagctgggggaggtcatcaatgaatctccatacttgtctcccaaatcttgttagcataacactatacggatgtacaaggttggagcatcttccattgatgagtcaactgcgtcatttgaaattccttacattacgatatttgagtgaagtggtgtatatggagaatagtattatcagcgcagaaggggttgcatcgacatcaggagggtcaagtcatggaggagcagatgaggacttggttttctttccatcccttgaaaagcttgagctttggagtatgccaaagttggaaggatggtggaaattagaatcagatatg

SEQ ID NO: 8 - protein, 333 aa, Spinacia tetrandra
  REGION 1..333: note = LRR domain protein sequence
RIDALSSNSTYLLSKVEYLRVLSLNNSDLEMLPSEIGNLLHLRYLDLSDNYRLSKLPTSITKLYNLQILKLRECRGLKTLPSDLRKLVNLRHLDIQGCKSLSHMPRGMNSMTSLHKLTGFVVSRKRNSWNRGSVSVGELGDLKNFSNHSHNMVIYVKKDAVDAREKGFLLKSEHLREIQYVWNHESEGTDADALLQGKNADVVLQGLQPHLNLRMLELRDYPGIRFPSWGRSSMNLHTCLPNLVSITLYGCTRLEHLPLMSQLRHLKFLTLRYLSEVVYMENSIISAEGVASTSGGSSHGGADEDLVFFPSLEKLELWSMPKLEGWWKLESDM

SEQ ID NO: 9 - RNA, 21 nt, synthetic construct
  source 1..21: mol_type = other RNA
tataaaccgggccgatgcctt

SEQ ID NO: 10 - RNA, 21 nt, synthetic construct
  source 1..21: mol_type = other RNA
ggcatcggcccggtttataag

SEQ ID NO: 11 - RNA, 21 nt, synthetic construct
  source 1..21: mol_type = other RNA
ataaaccgggccgatgccttc

SEQ ID NO: 12 - RNA, 21 nt, synthetic construct
  source 1..21: mol_type = other RNA
aggcatcggcccggtttataa

SEQ ID NO: 13 - RNA, 21 nt, synthetic construct
  source 1..21: mol_type = other RNA
cgatgcaaccccttctgcgct

SEQ ID NO: 14 - RNA, 21 nt, synthetic construct
  source 1..21: mol_type = other RNA
cgcagaaggggttgcatcgac

SEQ ID NO: 15 - RNA, 21 nt, synthetic construct
  source 1..21: mol_type = other RNA
tataaaccgggccgatgcctt

SEQ ID NO: 16 - RNA, 20 nt, synthetic construct
  source 1..20: mol_type = other RNA
cgcagaaggggttgcatcga

SEQ ID NO: 17 - RNA, 21 nt, synthetic construct
  source 1..21: mol_type = other RNA
ctaacggggtgtctggagcca

SEQ ID NO: 18 - RNA, 21 nt, synthetic construct
  source 1..21: mol_type = other RNA
gctccagacaccccgttagtc

SEQ ID NO: 19 - DNA, 280 bp, synthetic construct
  source 1..280: mol_type = other DNA
ggatttcccgcagtgacactgacattatcgaagcaacaatgaaggaacttgccaaacttttcccagacgaaattgcagctgatgggagcaaggctaagatcctcaaatatcatgttgtcaagactccaaggtctgtttataagacagttccagactgtgagccttgtcggccactgcaaagatcaccactagaaggtttctatttatctggtgattacacaaagcaaaaatatttggcttcaatggaaggtgctgttttatctgggaagttttgtgcaca

SEQ ID NO: 20 - DNA, 607 bp, synthetic construct
  source 1..607: mol_type = other DNA
agatgaggacttggttttctttccatcccttgaaaagcttgagctttggagtatgccaaagttggaaggatggtggaaattagaatcagatatgggtgagacacgagaagtagcagggtttcaaacacattcatattcgttccatcacctttctcatctgttaattacattttgtgataatttgagaaattttcctctctgtccgaaactggaagaatcaaactcccaactacaggaaatcagaaatctgaatcattcttttaccagtaagatcttcttttttattgtgtgtgtgtaaaaggttacatatttttatatatacctagtagctcctaactatctacaagtataactttgttcatggtataagtgcagaagacgaagttgagtccctccaatccttgcaatttgaccttgaaactatagaaattgcaacacataacttttctgatgataataagatcggagaaggcatcggcccggtttataagggtatacttcctgatgggcaagagatagaagttaagaggcttttaaggaactcgcgccaaagagatgcagagttcaaaaatgaaattttgatattagccaaggtccggcacaagaa

SEQ ID NO: 21 - DNA, 3801 bp, Spinacia tetrandra
  source 1..3801: mol_type = other DNA
atggatgtgtctgcaggcctttcactagtacaaactgtcctcgaattgttgggttctcccatttggacacagattcaatctttgttgggtgtggagtactcacagcttgacaaactcaaggccaccatgtccactattgaggctgtgctccttgatgctgaagatcaagagcagttgcatcaatatcgtcgtagccatgttgaactggataggcttcagaggcttaaggaagctctttatatagctgatgacttgttcgacaagattgccactcttgctcaacgcaaaaccctcataatgcgctttaataacaagctgcttaatgaggtatgccttttcttctcttcctctaataagcttcgttctgctttcactgggtctcgtgagatggagaacattagggagatgttgaatgatattgtcaaagatcatcgctatgactttggctccagacaccccgttagtctggatgcgaaacagctgagaatcaaagcaagggaaactcattcctttgtttgcgaggaagatatgatcattgggagggatgatgacaagcggaaagtgatagatatggtactaaacacttcttgtgaggagaaggatatttctgttgtcagcatagtggggattggggggctgggaaaaacaactctagcccgacttgtttataatgatgagaagataggtagtaaattctctctgaaaatgtgggtttgtgtctctgatagtttcaatgtcaaagatcttgttgctgaaatccttgcagccgtaaaccctagaagaaagcacgatgccctgagtatgagtatggaccagcttcagatgaaaattcgggacgaactggatgggaagatatacctccttgttttggatgatgtatggactgaagatcccattaaatggcatgagctgagaaaactattgattggaggtagaaacggtagcagaattctggtgactactcgctcaagtggggtggcgaaagttgttggaagctatcatattcataaattggaaggactctctaataaagagtcttgggatttgtttgagaggatgacacttgaaccaggacaacatcaaatgcaagagcacttggttaaaattggaaaagatattgtgagaaagtgtgcaaatgttccacttgctataagggtggttggaagtcttcttcgaggtcagggtgaaagtaggtggcaatatttgaaaaacactgacttggcaaatataccacaagatgagatcaatggtattttacctgttttgaagattagttattattatcttccgttccatttgaagagttgctttaagtattgttctgtattccccaaggattacaagattatcaaggaggatttgatcagtctctggatggcacaaggcttcatcatctcatcaaatgaagagagtttcgaaaatgcaggtgaggaatatttcaaacaattactccaaaggtgtttcttccaagatgtagaacgggccgagggtactgatgaaattctatcatgcaagatgcatgacttgattcatgaccttgctagtgaagtagcaggaacagaaatcgtgtcgtccaagtataatataagggggtttagcgagaaaactcgccatatatttattgatagagatgccgttaaagatatttatcgtcatttcaccaatatgaaaaggatgcgttcaatttttagaatagatgcgctttcttctaattcaacatatttgttatcaaaagtggaatatttaagggtcttaagcttgaataactctgatttggaaatgttgccgagtgagataggtaacttgttgcatctaagatatcttgacctatcagataattaccgtctctctaagttgcctacctccattaccaagctttacaacttgcaaatattgaagttacgtgagtgtcgtggtctaaaaactttgccaagcgatttgaggaaattagtaaatctaaggcacctagatatacaaggctgtaagagtttgtcgcatatgccccgggggatgaatagcatgacatctctgcataaactgacagggtttgtagtgagcagaaaaagaaatagctggaatcgggggtcagttagtgttggtgagctgggagatttgaaaaatttcagcaatcatagccacaatatggtaatttatgtcaaaaaagatgcagttgatgctagggaaaaggggtttctattaaaaagtgaacatttgagggaaattcagtatgtttggaatcatgagagtgaggggactgatgcagatgcattgctgcaagggaaaaatgcagatgtggtgctgcaagggctgcaaccccatcttaatctccgaatgttggagttgagggattatccaggaattaggttcccaagctgggggaggtcatcaatgaatctccatacttgtctcccaaatcttgttagcataacactatacggatgtacaaggttggagcatcttccattgatgagtcaactgcgtcatttgaaattccttacattacgatatttgagtgaagtggtgtatatggagaatagtattatcagcgcagaaggggttgcatcgacatcaggagggtcaagtcatggaggagcagatgaggacttggttttctttccatcccttgaaaagcttgagctttggagtatgccaaagttggaaggatggtggaaattagaatcagatatgggtgagacacgagaagtagcagggtttcaaacacattcatattcgttccatcacctttctcatctgttaattacattttgtgataatttgagaaattttcctctctgtccgaaactggaagaatcaaactcccaactacaggaaatcagaaatctgaatcattcttttaccaaagacgaagttgagtccctccaatccttgcaatttgaccttgaaactatagaaattgcaacacataacttttctgatgataataagatcggagaaggcatcggcccggtttataagggtatacttcctgatgggcaagagatagaagttaagaggcttttaaggaactcgcgccaaagagatgcagagttcaaaaatgaaattttgatattagccaaggtccggcacaagaatttggtgaaacttctaggattttgcttgcatggagaagaaaggattcttgtctatgagtttgttgccaacagaagccttgacaactttatatttgatccattgagtcgtcagtctatgcaatgggaaattcgttataagattatcaatggcatagctagagggattctatacctccatgaaggttccagactaatgatcatacatcgcaacctagaagcaggaagtgttctacttgatgaagatttttgtcccaagattgcagattttggtatggcatggctatttaacatagatcaaactcaaacagaggctagcagaatcgctggaaccttaggatatattgcacctgagtatgctacacaatatcaagtatcagtaaagtcagacgtatacagctttggtatactagtcctagagatagtaagcgggcagaggattacttcatttcattacgagggagaaaacctcacgacctctgcatggagaaactggaatgaaggcacagcttggaatctggtagatcctgtgttactaggcagctctagaaccgagatactgcgatgcatccatatcggtttgttgtgtgtccaagaaagccctgcagacaggccaactatgtctgaggttcatgttatgttgagtacgaattcggtcacccttgaagcacctttgcagcctgcattttgtacagatagccaaagtctaccc

SEQ ID NO: 22 - protein, 1287 aa, Spinacia tetrandra
MDVSAGLSLVQTVLELLGSPIWTQIQSLLGVEYSQLDKLKATMSTIEAVLLDAEDQEQLHQYRRSHVELDRLQRLKEALYIADDLFDKIATLAQRKTLIMRFNNKLLNEVCLFFSSSNKLRSAFTGSREMENIREMLNDIVKDHRYDFGSRHPVSLDAKQLRIKARETHSFVCEEDMIIGRDDDKRKVIDMVLNTSCEEKDISVVSIVGIGGLGKTTLARLVYNDEKIGSKFSLKMWVCVSDSFNVKDLVAEILAAVNPRRKHDALSMSMDQLQMKIRDELDGKIYLLVLDDVWTEDPIKWHELRKLLIGGRNGSRILVTTRSSGVAKVVGSYHIHKLEGLSNKESWDLFERMTLEPGQHQMQEHLVKIGKDIVRKCANVPLAIRVVGSLLRGQGESRWQYLKNTDLANIPQDEINGILPVLKISYYYLPFHLKSCFKYCSVFPKDYKIIKEDLISLWMAQGFIISSNEESFENAGEEYFKQLLQRCFFQDVERAEGTDEILSCKMHDLIHDLASEVAGTEIVSSKYNIRGFSEKTRHIFIDRDAVKDIYRHFTNMKRMRSIFRIDALSSNSTYLLSKVEYLRVLSLNNSDLEMLPSEIGNLLHLRYLDLSDNYRLSKLPTSITKLYNLQILKLRECRGLKTLPSDLRKLVNLRHLDIQGCKSLSHMPRGMNSMTSLHKLTGFVVSRKRNSWNRGSVSVGELGDLKNFSNHSHNMVIYVKKDAVDAREKGFLLKSEHLREIQYVWNHESEGTDADALLQGKNADVVLQGLQPHLNLRMLELRDYPGIRFPSWGRSSMNLHTCLPNLVSITLYGCTRLEHLPLMSQLRHLKFLTLRYLSEVVYMENSIISAEGVASTSGGSSHGGADEDLVFFPSLEKLELWSMPKLEGWWKLESDMGETREVAGFQTHSYSFHHLSHLLITFCDNLRNFPLCPKLEESNSQLQEIRNLNHSFTKDEVESLQSLQFDLETIEIATHNFSDDNKIGEGIGPVYKGILPDGQEIEVKRLLRNSRQRDAEFKNEILILAKVRHKNLVKLLGFCLHGEERILVYEFVANRSLDNFIFDPLSRQSMQWEIRYKIINGIARGILYLHEGSRLMIIHRNLEAGSVLLDEDFCPKIADFGMAWLFNIDQTQTEASRIAGTLGYIAPEYATQYQVSVKSDVYSFGILVLEIVSGQRITSFHYEGENLTTSAWRNWNEGTAWNLVDPVLLGSSRTEILRCIHIGLLCVQESPADRPTMSEVHVMLSTNSVTLEAPLQPAFCTDSQSLPDMLREWSTTNSQVSDDESEP

(19) United States
(12) Patent Application Publication
(10) Pub. No.: US 2025/0243505 A1
(43) Pub. Date: Jul. 31, 2025

(54) Genes From Spinacia Tetrandra Encoding a Protein Providing Resistance Against Peronospora Farinosa and Spinach Plants Comprising These Genes

(71) Applicant: Bejo Zaden B.V., Warmenhuizen (NL)

(72) Inventors: Stefanus Johannes Kaandorp, Warmenhuizen (NL); Raimon Jozef Laan, Warmenhuizen (NL); Klaas Schotanus, Warmenhuizen (NL); Roelof Marinus Veenstra, Warmenhuizen (NL); Albertus Johannes Maria Schrijver, Warmenhuizen (NL)

(21) Appl. No.: 18/880,934

(22) PCT Filed: Jul. 4, 2023
(86) PCT No.: PCT/EP2023/068358

§ 371 Date: Jan. 3, 2025

(30) Foreign Application Priority Data
Jul. 5, 2022 (WO) PCT/EP2022/068558

(51) Int. Cl. C12N 15/82; C12N 9/22; C12N 15/11; C12Q 1/6895

(52) U.S. Cl. CPC C12N 15/8282; C12N 9/222; C12N 15/111; C12N 15/8205; C12Q 1/6895; C12N 2310/20; C12Q 2600/13

(57) ABSTRACT

The present invention relates to Spinacia tetrandra genomic DNA including a first or a second genomic DNA fragment, wherein the first or the second genomic DNA fragments include genes encoding proteins providing resistance against the plant pathogen Peronospora farinosa. The present invention further relates to spinach plants being resistant to the plant pathogen Peronospora farinosa, including the first or the second genomic DNA fragments. The present invention further relates to methods for providing and to methods for identifying spinach plants being resistant to the plant pathogen Peronospora farinosa. The present invention also relates to the use of one or more nucleic acid sequences or amino acid sequences for providing, or identifying, plants resistant to the plant pathogen Peronospora farinosa.

### CROSS-REFERENCE TO RELATED APPLICATIONS

This application is the United States national phase of International Patent Application No. PCT/EP2023/068358 filed Jul. 4, 2023, and claims priority to International Patent Application No. PCT/EP2022/068558 filed Jul. 5, 2022, the disclosures of which are hereby incorporated by reference in their entireties.

### SEQUENCE LISTING

The Sequence Listing associated with this application is filed in electronic format via patent Center and is hereby incorporated by reference into the specification in its entirety. The name of the file containing the Sequence Listing is 2408396.xml. The size of the file is 71,331 bytes, and the file was created on Dec. 5, 2024.

### BACKGROUND OF THE INVENTION

### Field of the Invention

The present invention relates to Spinacia tetrandra genomic DNA comprising a first or a second genomic DNA fragment, wherein the first or the second genomic DNA fragments comprise genes encoding proteins providing resistance against the plant pathogen Peronospora farinosa. The present invention further relates to spinach plants being resistant to the plant pathogen Peronospora farinosa, comprising the first or the second genomic DNA fragments. The present invention further relates to methods for providing and to methods for identifying spinach plants being resistant to the plant pathogen Peronospora farinosa. The present invention also relates to the use of one or more nucleic acid sequences or amino acid sequences for providing, or identifying, plants resistant to the plant pathogen Peronospora farinosa.

Spinach is commercially grown worldwide for its attractive and nutritious leaves. In 2018, production of spinach was close to 26 million tons worldwide. Spinach (Spinacia oleracea or S. oleracea) is a member of the Amaranthaceae family, subfamily Chenopodioideae. Other well-known family members include quinoa and beet. The latter is a cultivated plant of major importance for agriculture with sugar beet, red beet and Swiss chard as examples.

Regarding nutritional value, while providing only relatively small amounts of calories (23 for 100 grams of cooked spinach), spinach is a rich source of vitamins A, B2 (or folate), B6, C, E and K and, additionally, magnesium, manganese, calcium, potassium, iron and dietary fibre.

Spinach flowering is induced by (long) day lengths and under optimal conditions spinach can reach even up to 4 generations in a year with a life cycle from seed to new harvest completed within 3 months. A bottleneck in spinach production can be caused by seed dormancy.

Spinach is a wind pollinator and its pollen can reach far. A line is considered male if it converts from female or mixed flowering to (all) male flowering within a week. Female lines stay so for at least three weeks without producing any pollen. Hybrids of spinach can be produced making use of plants which have a female flowering phase and plants which have a male flowering phase as pollinator. Before the female plants develop male flowers, all female flowers are fertilized by the male plant. The setting of seeds occurs rapidly within 3 days and after that the ripening of the seed takes approximately a month.

Under optimal conditions commercial elite spinach lines are grown and harvested within 25 days.

Breeding resulted in spinach plants which are rapidly growing without premature flowering. Older varieties tend to have narrower leaves and have a stronger, somewhat bitter taste; newer varieties have broader leaves and a milder taste. Also, recent types have little tendency for bolting in warm conditions and therefore will not prematurely flower and produce seeds.

Spinach is cultivated for the leaves. Commercial spinach can have round leaves of a dark green color. Leaf morphology is of interest to spinach breeders. A significant share of the market of cultivated spinach is the early harvested baby leaf spinach. For spinach growers it is important that the leaves stand straight up which facilitates easy harvest and dark green color is desirable.

Spinach originated from middle Asia but it is now produced all over the world. Traditional areas where spinach was grown as a crop are Europe and Northern America, however contemporarily the biggest volume of spinach is produced in China. Spinach is produced for the food processing industry (canned or frozen spinach) as well as for the fresh market, where especially baby leaf spinach is in demand. Breeders develop lines with characteristics best suited for the location or the purpose.

An important development in the production and sales of fresh spinach was the introduction of bagged spinach. For this application the desired leaf morphology is such that the leaves are not too closely packed together that are found in the partly savoyed types.

Basic types of spinach presently on the market are:
A savoy type with dark green, curly and crinkly leaves (mainly for the fresh market);
A flat, or smooth, leaf spinach with broad, smooth leaves that can be cleaned easily. This type is used for industry (canned or frozen spinach, as well as processed food and baby food);
Semi savoy is an intermediate type of spinach with a comparable texture as the savoy type but as easy to clean as the smooth type of spinach. It is cultivated both for fresh market and industry.
An oriental type which is heat tolerant, has long petioles, pointed leaves with several side lobes and, as plant, has an upright growth.

Most spinach is produced at high plant densities for fresh market production which creates an ideal environment for disease development. Additionally, there is an increasing demand to produce organic vegetables because consumers are increasingly looking for vegetables that are obtained without the use of pesticides, fungicides, insecticides and without chemical treatment of seeds. The challenge here is that in the absence of pesticides, fungicides, insecticides spinach plant are to susceptible to plant diseases. Accordingly, there is a need in the art for spinach cultivars that encompass genetically, preferably naturally occurring, encoded resistances against pathogens.

The most common pathogens causing diseases in spinach are Peronospora, Fusarium, Stemphyllium, Colletotrichum, Cercospora and Cucumber Mosaic Virus. A major disease in spinach is downy mildew caused by the oomycete pathogen Peronospora farinosa or Peronospora effusa (also designated as P. farinosa f. sp. spinaciae or abbreviated Pfs). The short lifecycle of Pfs results in rapid multiplication of the pathogen on susceptible cultivars. At first, small pale yellow irregular spots appear on the upper surface of the leaves and a purple downy growth on the lower surface of the spots. Spores develop on the leaves 9-12 days after first infection and are spread by wind and splashes of water. Infected leaves are no longer attractive for consumption and prone to other, secondary (microbial) infections.

One way to combat downy mildew is to spray the plants with fungicide. This approach is highly undesirable due to its heavy impact on the environment and, additionally, it is costly and labor intense. Moreover, half of the agriculturally produced spinach is meant for the organic market and making use fungicides not suitable for combating downey mildew.

Peronospora farinosa is a pathogen that rapidly overcomes, or breaks, resistance. Within 2 to 3 years, newly introduced resistance genes are observed to be bypassed by the pathogen necessitating a constant demand to identify new sources of resistance. To date, seventeen official races have been described by the International Working group on Peronospora effusa/farinosa/Pfs (IWGP). Since only a limited set of Resistance to Peronospora (RPF) genes have been identified that originate from S. oleracea, wild relatives of spinach are a potential interesting source of novel and alternative RPF genes.

Effector triggered immunity (ETI) is a part of plant immune system, NB-LRR proteins form a class of proteins that can trigger ETI. NB-LRR proteins obtain their name from a central Nucleotide-Binding domain and a C-terminal Leucine Rich Repeat domain. Many plant disease resistant proteins are NB-LRR proteins wherein NB-LRR proteins recognize the pathogen effector and activate host defenses. The C-terminal LRR domains of NB-LRR proteins are highly irregular, have varying lengths and differ in the number of LRR repeats. The LRR domain is involved in pathogen recognition and mutations in the C-terminal half of the LRR domains have been shown to influence recognition specificity. LRR domains contain patches with epitopes involved in effector binding. Recognition of the pathogen effector through the LRR domain transduces a signal to the rest of the protein. WO2018059651 and related patent literature documents disclose WOLF genes encoding proteins of the CC-NB-LRR family providing for resistance against Peronospora farinosa in spinach plants.

### Description of Related Art

Considering that a significant part of the spinach production is grown organically there is a high demand for spinach varieties having resistance to all known Peronospora farinosa races (presently Pfs 1-19). However, in fields with fully resistant, i.e., resistant to Pfs 1 to 19, spinach cultivars the problem encountered by growers changes from Peronospora farinosa to other pathogens that can affect spinach. An example of such a pathogen is the fungus Stemphylium, the causal agent of Stemphylium leaf spot. Two species of Stemphylium have been described to cause disease on spinach that include Stemphylium beticola (previously Stemphylium botryosum) and Stemphylium vesicarium. In recent years, S. vesicarium is the most prevalent species of the two species.

Stemphylium vesicarium produces typical conidiospores that germinate on leaf surfaces and cause small necrotic lesions on spinach leaves with brown rings. Leaf spots can significantly reduce the quality and yield of spinach especially for the fresh market. Varietal differences in response to S. vesicarium have been observed.

Another pathogen that affects spinach production is the virus Cucumber Mosaic Virus (CMV) the causal agent of spinach blight. CMV belongs to the family of Bromoviridae and the genus Cucumovirus and exhibits a broad host range of 1200 plant species in over 100 plant families. Economically important crops that can suffer from CMV infection include cucurbits, pepper, lettuce, celery, tomato, and beans. Genetically encoded resistance to CMV can prevent spread of CMV especially when crop rotations with susceptible crops is normal practice. Symptoms on spinach include yellowing of the leaves, distortion of the crown leaves, rolling leaves, stunting and dying plants. Leaves with yellowing are unsuitable to sell for fresh market spinach. Therefore, genetic resistance to CMV is a welcome addition for disease resilience in spinach plants.

As indicated, there is a rapid adaptation of Peronospora farinosa to the genetically encoded resistance. New Peronospora farinosa isolates continuously emerge capable of evading host recognition thereby bypassing resistance. To counter this rapid adaptation breeders either search for new resistance genes or combine, or stack, known resistances, i.e., introduce two or more different Peronospora farinosa resistance genes into one plant to increase the chance that a plant can overcome Peronospora infection or reduce the probability that the 19 pathogen races that are currently recognized, overcome the resistance. There is thus a need in the art to provide new and alternative genetically encoded resistances against Peronospora farinosa to enable breeders to develop novel spinach cultivars that are resistant to the plant pathogen Peronospora farinosa. It is an object of the present invention, amongst other objects, to meet the above need in the art.

### SUMMARY OF THE INVENTION

This object, amongst other objects, is met by the present invention as outlined in the appended claims.

Specifically, the above object, amongst other objects is met, according to a first aspect by providing Spinacia tetrandra genomic DNA, comprising
a first genomic DNA fragment having the nucleic acid sequence of Seq ID No. 1 or a nucleic acid sequence having at least 90% identity with Seq ID No. 1, or
a second genomic DNA fragment having the nucleic acid sequence of Seq ID No. 2 or a nucleic acid sequence having at least 90% identity with Seq ID No. 2,
   wherein the first or second genomic DNA fragment comprises a gene encoding a protein providing resistance against the plant pathogen Peronospora farinosa.

Within the context of the present invention, “a spinach reference genome” or “the spinach reference genome” refers to the spinach genome published by Cai, X., Sun, X., Xu, C. et al. Genomic analyses provide insights into spinach domestication and the genetic basis of agronomic traits. Nat Commun. 12, 7246 (2021). https://doi.org/10.1038/s41467-021-27432-z. Based on this publicly available spinach genome, a skilled person will readily be able to identify the corresponding positions in any spinach genome, for example by aligning the sequence of the recited genomic fragments either completely or partly.

More specifically the above object, amongst other objects is met, by providing Spinacia tetrandra genomic DNA, comprising
a first genomic DNA fragment having the nucleic acid sequence of Seq ID No. 1 or a nucleic acid sequence having at least 90%, 91%, 92%, 93%, 94%, 95%, 96%, 98%, or 99% identity with Seq ID No. 1, or
a second genomic DNA fragment having the nucleic acid sequence of Seq ID No. 2 or a nucleic acid sequence having at least 90%, 91%, 92%, 93%, 94%, 95%, 96%, 98%, or 99% identity with Seq ID No. 2,
   wherein the first or second genomic DNA fragment comprises a gene encoding a protein providing resistance against the plant pathogen Peronospora farinosa.

Within the context of this invention sequence identity is understood as consecutive nucleic acid or amino acid sequence identity over the entire sequence using commonly known alignment tools such as BLAST (https://blast.ncbi.nlm.nih.gov/Blast.cgi).

Further, the above object, amongst other objects is met, according to another aspect, by providing Spinacia tetrandra genomic DNA, comprising:
the first genomic DNA fragment comprises a gene encoding a first resistance protein providing resistance against the plant pathogen Peronospora farinosa, wherein the first resistance protein comprises the amino acid sequence of SEQ ID No. 5 or an amino acid sequence having at least 85%, 86%, 87%, 88%, 89%, 89%, 90%, 91%, 92%, 93%, 94%, 95%, 96%, 97%, 98%, or 99% identity with SEQ ID No 5; or
the second genomic DNA fragment comprises a gene encoding a second resistance protein providing resistance against the plant pathogen Peronospora farinosa, wherein the second resistance protein comprises the amino acid sequence of SEQ ID No. 6 or a sequence having at least 85%, 86%, 87%, 88%, 89%, 89%, 90%, 91%, 92%, 93%, 94%, 95%, 96%, 97%, 98%, or 99% identity with SEQ ID No. 6 and/or
the second genomic DNA fragment comprises a gene encoding a third resistance protein providing resistance against the plant pathogen Peronospora farinosa, wherein the third resistance protein comprises the amino acid sequence of SEQ ID No. 22 or a sequence having at least 85%, 86%, 87%, 88%, 89%, 89%, 90%, 91%, 92%, 93%, 94%, 95%, 96%, 97%, 98%, or 99% identity with SEQ ID No. 22.

Further, the above object, amongst other objects is met, according to another aspect, by providing Spinacia tetrandra genomic DNA, wherein:
the first genomic DNA fragment comprises the first cDNA nucleic acid sequence of SEQ ID No. 3 or a sequence having at least 90% identity with SEQ ID No. 3 such as at least 91%, 92%, 93%, 94%, 95%, 96%, 98%, or 99% identity; or
the second genomic DNA fragment comprises the second cDNA nucleic acid sequence of SEQ ID No. 4 or a sequence having at least 90% identity with SEQ ID No. 4 such as at least 91%, 92%, 93%, 94%, 95%, 96%, 98%, or 99% identity; and/or
the second genomic DNA fragment comprises the third cDNA nucleic acid sequence of SEQ ID No. 21 or a sequence having at least 90% identity with SEQ ID No. 21 such as at least 91%, 92%, 93%, 94%, 95%, 96%, 98%, or 99% identity.

According to another aspect the above object, amongst other objects is met by providing a protein comprising the amino acid sequence of SEQ ID No. 5, 6, or 22 or a protein comprising an amino acid sequence having at least 85%, 86%, 87%, 88%, 89%, 89%, 90%, 91%, 92%, 93%, 94%, 95%, 96%, 98%, or 99% identity with SEQ ID No. 5, 6 or 22.

According to yet another aspect the above object, amongst other objects is met by providing a cDNA comprising the nucleic acid sequence of SEQ ID No. 3, 4, or 21 or a cDNA comprising the nucleic acid sequence having at least 90%, 91%, 92%, 93%, 94%, 95%, 96%, 98%, or 99% of SEQ ID No. 3, 4, or 21.

According to another embodiment, the above object, amongst other objects is met by providing a spinach plant which is resistant to the plant pathogen Peronospora farinosa, comprising:
a first or a second genomic DNA fragment as defined above, and/or
a first or a second resistance protein and/or third resistance protein as defined above, and/or
a first or a second cDNA sequence and/or third cDNA sequence as defined above.

Alternatively, the present invention relates to a spinach plant which is resistant to the plant pathogen Peronospora farinosa, comprising:
a first and a second genomic DNA fragment as defined above, and/or
a first and a second resistance protein and/or third resistance protein as defined above, and/or
a first and a second cDNA sequence and/or third cDNA sequence as defined above.

According to a preferred embodiment, the above object, amongst other objects is met by providing a spinach plant as defined above, wherein the plant is at least resistant to the plant pathogens Peronospora farinosa races Pfs 10 to Pfs 19.

The present inventors have surprisingly discovered that by introducing a genomic fragment of Spinacia tetrandra on chromosome 4 an additional resistance providing genomic fragments can be introduced on chromosome 3. This opened the possibility to introduce further resistances into the plant.

According to an even more preferred embodiment, the above object, amongst other objects is met by providing a spinach plant as defined above, wherein the plant is further resistant to the plant pathogens Stemphylium vesicarium and/or CMV.

According to another aspect the above object, amongst other objects is met by providing a spinach plant as defined above, wherein the resistance is obtained, is obtainable, or is from deposit NCIMB 43993 for the first Spinacia tetrandra genomic fragment and/or deposit NCIMB 43994 for the second Spinacia tetrandra genomic fragment.

Seeds of plants according to the invention, Spinacia oleracea 2130195, deposit NCIMB 43993 and Spinacia oleracea 2025195, deposit NCIMB 43994, were deposited at NCIMB Limited, Craibstone Estate, 35 Ferguson Building, Bucksburn, Aberdeen AB21 9YA, United Kingdom on Jun. 13, 2022.

Spinacia tetrandra and Spinacia turkestanica are wild relatives of the contemporary. Morphologically they resemble ancient spinach Spinacia oleracea. They are also either male or female with pointy leaves with sharp angles. There is however, a problem with using Spinacia tetrandra and material derived of it in the seed industry. S. tetrandra yields large, clustered and sharp seeds. This is highly undesired in seed production because of two reasons:
(i) it is difficult to obtain even coating of sharp seed and
(ii) sharp seed is not compatible with automatic seed sowing machines.
   Within the context of this invention, sharp seed is defined as non-round seed, having spikes and a tendency to cluster. This in contrast with agriculturally elite S. oleracea that predominantly produces round, or rounded, seeds without spikes an no tendency to form clusters. There is thus a need in the field to provide spinach plants that produce non-sharp seeds within a seed lot.

Accordingly, the present invention, according to an especially preferred embodiment, provides a spinach plant as defined above, wherein the plant produces seeds comprising at most 25%, preferably 20%, more preferably 10% or even more preferably 5% sharp seeds.

According to another aspect the present invention relates to a method for providing a spinach plant being resistant to the plant pathogen Peronospora farinosa, wherein the method comprises the step of introducing:
a first or a second genomic DNA fragment as defined above, or
a first, second and/or third cDNA sequence as defined above;
   operably connected with appropriate expression and translation sequences, into a pathogen Peronospora farinosa susceptible spinach plant.

More specifically the above method comprises the use of Agrobacterium and/or CRISPR Cas.

Even more specifically in the method above, the first and a second genomic DNA fragment, is obtainable, is obtained, or is from the deposit number NCIMB 43993 and NCIMB 43994, respectively.

According to yet another aspect the present invention relates to a method for identifying a spinach plant being resistant to Peronospora farinosa,

wherein the method comprises the steps:
isolating or providing plant material of the spinach plant,
detecting in the plant material:
   a first or a second genomic DNA fragment as defined above and/or
   a first, a second and/or third resistance protein as defined above, and/or
   a first, a second and/or third cDNA sequence as defined above.

Additionally, the present invention further relates to use of one or more nucleic acid sequences selected from the group consisting of: SEQ ID No. 1, SEQ ID No. 2, SEQ ID No. 3, SEQ ID No. 4 and SEQ ID No. 21, and/or use of one or more amino acid sequences selected from the group consisting of: SEQ ID No. 5, SEQ ID No. 6, SEQ ID No. 8 and SEQ ID No. 22 for identifying or providing plants resistant to plant pathogen Peronospora farinosa, preferably races Pfs 10 to Pfs 19.

### BRIEF DESCRIPTION OF THE DRAWINGS

The present invention will be further described by the examples below and the accompanying figures wherein:

FIG. 1: shows S. tetrandra seeds;

FIG. 2: shows S. tetrandra x S. oleracea and backcrossed to S. oleracea seeds;

FIG. 3: shows seeds of NCIMB 43994, i.e., S. oleracea with small introgressions of S. tetrandra;

FIG. 4: shows the present first and second S. tetrandra genomic fragments. Abbreviations used in the figure: TSS—Transcription Start Site; Start codon; Exons (CDSi, gray); Last exon with stop codon (blue, CDSI); Terminator is behind the stop codon; polyA tale. Within the first S. tetrandra genomic fragment (SEQ ID No. 1) and the second S. tetrandra genomic (Seq ID No 2) a smaller fragment was selected where a potential gene was localized, and visualized. Both first and second genomic fragments comprise of 7 exons each, including the start and the stop codon;

FIG. 5: shows the second S. tetrandra accession's cDNA: as predicted by Augutsus, Seq ID No. 4 (green) compared to results of the sequencing obtained for splicing variant 1, Seq ID No 21 (yellow).

### EXAMPLE 1. PERONOSPORA FARINOSA—DISEASE TRIAL

### DESCRIPTION OF THE INVENTION

Resistance to Peronospora farinosa f.sp. spinaciae (synonym P. effusa [hereafter Pfs]) was tested in a qualitative disease assay. In short, 10 to 14 days after untreated seed were sown in soil, a minimum of 8 plants were inoculated with a spore suspension of a single Pfs race or isolate. Pfs was maintained on a living susceptible host plant, for example, Viroflay or Blight or plant material with spores stored for a maximum of 1 year at −20° C. Inoculated plants were incubated under plastic at high humidity (80-100%) and at a temperature ranging from 16° C.-20° C. After 24 hours, plastic was removed and plants were assessed at 9 to 12 days after inoculation. When sporulation was observed on the cotyledons or true leaves a plant was considered susceptible and when no sporulation was observed a plant was considered resistant.

A differential set as described in Table 1 is included in each disease trial under the same environmental conditions to confirm the race. This differential set for Pfs was developed by the International Working Group on Peronospora farinosa (IWGP) and can be found on the website of the International Seed Federation (ISF). This differential set that consists of spinach varieties and near-isogenic lines (NILs) is used to determine the Pfs race. In this table “−” indicates resistance (no sporulation), “+” indicates susceptibility (sporulation), “(−)” indicates intermediate resistance (sparse sporulation on the tips of cotyledons), “n.t.” indicates that the current strain was not tested. Seeds of this differential set and Pfs races can be obtained at Naktuinbouw (P.O. Box 40, NL-2370 AA, Roelofarendsveen, Netherlands, naktuinbouw.com).

TABLE 1
IWGP Spinach differential set for Pfs. Where “−” is resistant, “+” is susceptible
and “(−)” indicates intermediate resistance.

| | Race Pfs | | | | | | | | | | | | | | | | | | |
|---|---|---|---|---|---|---|---|---|---|---|---|---|---|---|---|---|---|---|---|
| Variety/NIL | 1 | 2 | 3 | 4 | 5 | 6 | 7 | 8 | 9 | 10 | 11 | 12 | 13 | 14 | 15 | 16 | 17 | 18 | 19 |
| Viroflay | + | + | + | + | + | + | + | + | + | + | + | + | + | + | + | + | + | + | + |
| NIL5 | − | − | + | + | + | + | + | + | + | + | + | + | + | + | + | + | + | + | + |
| NIL3 | − | + | − | + | − | + | + | − | − | + | − | − | + | − | + | − | + | + | − |
| NIL4 | − | − | − | − | + | + | + | + | + | + | + | + | + | + | − | + | + | + | + |
| NIL6 | − | + | − | − | − | + | − | + | + | + | − | + | (−) | + | − | − | + | − | + |
| NIL1 | − | − | − | − | − | − | − | + | − | + | − | + | − | + | − | − | + | − | + |
| NIL2 | − | − | − | − | | − | − | − | | − | + | + | + | + | − | + | + | + | + |
| Pigeon | − | − | − | − | − | − | − | − | − | − | − | − | − | + | − | + | + | + | + |
| Caladonia | − | − | − | − | − | − | | − | − | − | − | − | − | − | + | − | + | + | − |
| Meerkat | − | − | − | − | | − | − | − | − | − | − | − | − | − | − | + | (−) | + | + |
| Hydrus | − | − | − | − | − | | − | − | | − | − | − | − | − | − | − | − | − | + |

TABLE 2
Resistance pattern of spinach plants according to the invention.

| Race Pfs | Deposit NCIMB 43994 | Deposit NCIMB 43993 |
|---|---|---|
| 1 | − | n.t. |
| 2 | − | − |
| 3 | − | n.t |
| 4 | − | − |
| 5 | − | n.t. |
| 6 | − | n.t. |
| 7 | − | n.t. |
| 8 | − | n.t. |
| 9 | − | n.t. |
| 10 | − | − |
| 11 | − | − |
| 12 | − | − |
| 13 | − | − |
| 14 | − | − |
| 15 | − | − |
| 16 | − | − |
| 17 | − | − |
| 18 | − | − |
| 19 | − | − |

Where “−” is resistant and “+” is susceptible and “n.t.” is not tested.

### EXAMPLE 2. STEMPHYLIUM—DISEASE TRIAL

Resistance to Stemphylium vesicarium was tested in a qualitative disease assay. In short, 7 to 10 days after untreated seed were sown in soil, a minimum of 20 plants per line was transplanted in pots. Plants were inoculated between 14 and 20 days after sowing when the first true leaves were fully expanded. S. vesicarium is maintained in glycerol at −80° C. and multiplied on potato dextrose agar (PDA). Spores were harvested and counted resulting in a spore suspension with a concentration of 1*10⁴ spores/mL. Inoculated plants were incubated under plastic at high humidity (80-100%) and at a temperature ranging from 20° C.-22° C. After 24 hours plastic was removed and plants were assessed at 4 days after inoculation. When leaf spots were observed on the true leaves a plant was considered susceptible and when no leaf spots were observed a plant was considered resistant.

| Variety | Score |
|---|---|
| Deposit NCIMB 43993 | R |
| Deposit NCIMB 43994 | R |
| Responder | S |
| Patton | S |

### EXAMPLE 3. CMV—DISEASE TRIAL

Resistance to CMV was tested in a qualitative disease assay. In short, 7 to 10 days after untreated seed were sown in soil, a minimum of 20 plants per line was transplanted in pots. Plants were inoculated between 14 and 20 days after sowing when the first true leaves were fully expanded. CMV is maintained as lyophilized spinach leafs at 4° C. CMV is firstly mechanically inoculated on Nicotiana benthamiana followed by multiplication on a susceptible spinach variety. Spinach plants were assessed 10 days post inoculation. Plants with leaf yellowing were considered susceptible whereas plant without leaf yellowing were considered resistant.

| Variety | Score |
|---|---|
| Deposit NCIMB 43994 | R |
| Responder | S |

### EXAMPLE 4. SIGNIFICANTLY LESS SHARP SEEDS

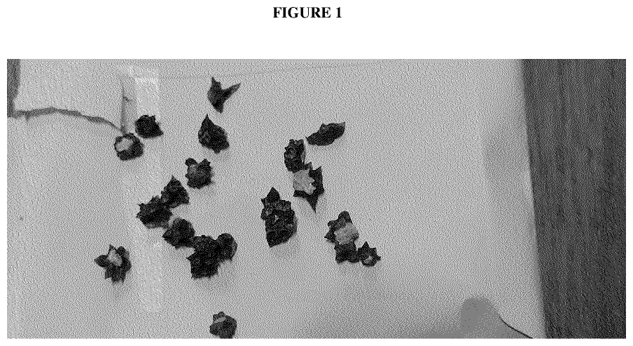

Spinacia tetrandra has sharp seed—FIG. 1. This type of seed (sharp seed) has the tendency to form clusters, which is undesired. Sharp seed is problematic because it is difficult to obtain an even layer of seed coating and it is hard to work with sharp seed in automatic seeding machines.

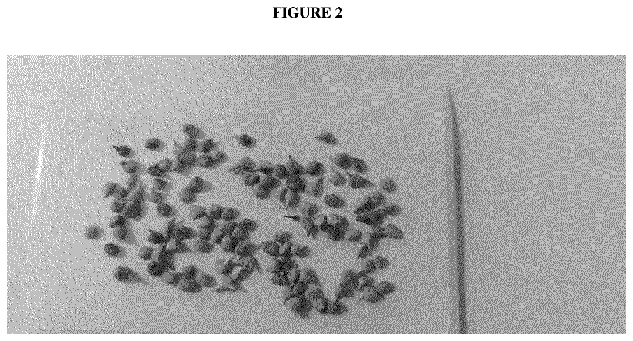

S. tetrandra was crossed with S. oleracea and three times backcrossed with S. oleracea—FIG. 2. Evaluation by visual inspection revealed that the seed lot resulting from this cross displayed 50% less sharp seed as compared to the S. tetrandra starting material.

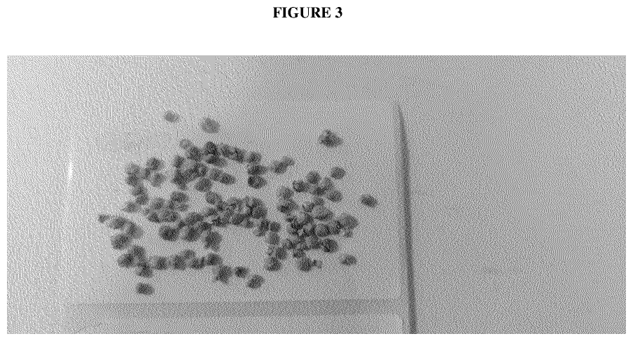

Plants of the Deposit NCIMB 43994—FIG. 3. These plants have only a relatively small introgression of S. tetrandra and their genome is predominantly S. oleracea. By visual inspection it was assessed that only 5% of the seed lot has sharp seeds and in the case that the seed was sharp it was significantly less sharp than the seeds of S. tetrandra (starting material). Plants of the deposit NCIMB 43993 showed similar results with respect to seed morphology as those of NCIMB 43994 (results not shown).

### EXAMPLE 5. SPINACIA TETRANDRA GENOMIC DNA SEQUENCES, CDNA AND PROTEIN

Based on QTL analysis, two S. tetrandra accessions that provided resistance against Peronospora farinosa have been identified and sequenced with Illumina technology. The sequencing data has been mapped against the S. oleracea reference genome and de novo genome assemblies were performed. Previously performed marker analysis resulted in the identification of a QTL and this QTL is located at chromosome 4 at (3468181 . . . 3487380) of the S. oleracea reference genome. Within the span of the QTL a highly interesting gene coding for a putative resistance protein was observed.

Comparative genomics between the S. oleracea reference genome and the two de novo S. tetrandra genomes resulted in the identification of one chromosomal region of each S. tetrandra de novo genome assembly. In the first S. tetrandra accession the genomic fragment is SEQ ID No. 1 of 13.5 kb; in the second S. tetrandra accession the genomic fragment is SEQ ID No. 2 of 17.5 kb.

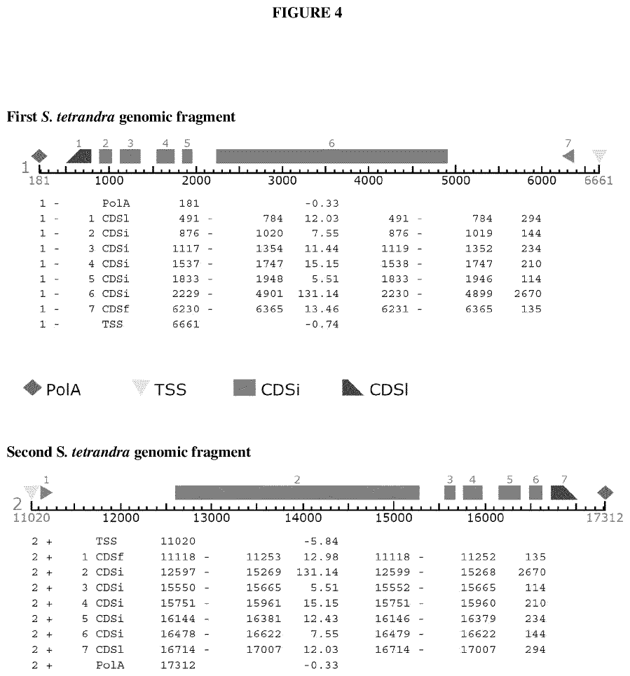

Softberry software was used to visualize the first genomic fragment (nucleic acid sequence according to SEQ ID No. 1) and the second genomic fragment (nucleic acid sequence according to SEQ ID No. 2). Within these fragments a smaller fragment was selected where a potential gene was localized, and visualized—FIG. 4. For both fragments the gene identified has a similar architecture, namely: 7 exons each, including the start and the stop codon.

Based on BlastN, the gene coding for a putative resistance protein was identified on the chromosomal fragment and the coding sequence of the gene of interest was predicted with Augustus. In the first S. tetrandra accession the cDNA is SEQ ID No. 3 of 3693 bp resulting a protein sequence of 1230 amino acids (SEQ ID No. 5). The cDNA of the second S. tetrandra accession is SEQ ID No. 4 (3693 bp) resulting in a protein sequence of 1230 amino acids (SEQ ID No. 6).

The coding sequences of the present resistance genes of both S. tetrandra accessions show 97.3% and 97.3% homology to the S. oleracea reference genome, respectively. Alignments showed that the putative resistance protein underwent positive selection in the S. tetrandra resistant accessions resulting in Peronospora farinosa resistance. The protein sequences of the putative resistance gene of both S. tetrandra accessions show respectively 95.1% and 95.2% homology to the S. oleracea reference genome, respectively.

To confirm the predictions of the gene sequences (i.e., Seq ID No 4, cDNA and Seq ID No 6, protein) of the second S. tetrandra accession, RT-PCR reactions were carried out.

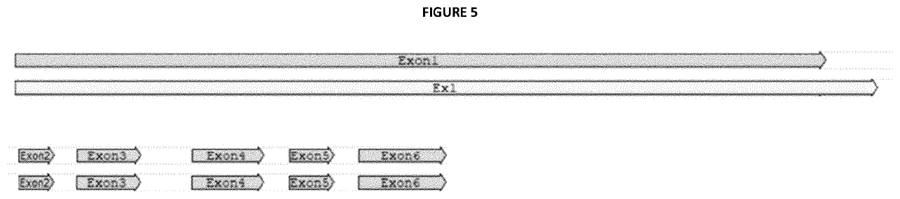

RNA was isolated with the innuPREP plant RNA kit (Analytik Jena), subsequently cDNA was synthesized with the First strand cDNA synthesis kit (NEB). Primers pairs were designed in the August predicted CDS, and after the PCR reaction this resulted in two distinct bands on the gel, which suggest the presence of two splicing variants. To confirm this result, the PCR products were sequenced with Nanopore sequencing and this resulted in the sequence of the first splice variant. In FIG. 5, the predicted cDNA sequence originating, Seq ID No 4 (green) is compared to results of the sequencing obtained for splicing variant, Seq ID No. 21 (yellow), both originating from the second S. tetrandra accession. It can be observed that Exon 1 differs between the prediction (Augustus) and the splice variant 1. In the case of the splice variant 1, the Exon 1 is longer with 2857 bp, The other exons were predicted correctly and their lengths are the same between the prediction (Augustus) and the splice variant 1.

The cDNA sequence of splice variant 1 originating from the second S. tetrandra accession is referred to as Seq ID No. 21. The protein that is the translation of said cDNA is referred to as Seq ID No. 22 (originating from the second S. tetrandra accession).

TABLE 3
The second S. tetrandra accession's cDNA as predicted
by Augustus (Seq ID No 4.) compared with the results of
the sequencing of splice variant 1 (Seq ID No. 21).

| | Start (bp) | Stop (bp) | Length (bp) |
|---|---|---|---|
| Augustus | | | |
| Exon1 | 1 | 2688 | 2687 |
| Exon2 | 2969 | 3083 | 114 |
| Exon3 | 3169 | 3380 | 211 |
| Exon4 | 3563 | 3801 | 238 |
| Exon5 | 3898 | 4042 | 144 |
| Exon6 | 4134 | 4427 | 293 |
| Splice variant 1 | | | |
| Exon1 | 1 | 2858 | 2857 |
| Exon2 | 2969 | 3083 | 114 |
| Exon3 | 3169 | 3380 | 211 |
| Exon4 | 3563 | 3801 | 238 |
| Exon5 | 3898 | 4042 | 144 |
| Exon6 | 4134 | 4427 | 293 |

### EXAMPLE 6. THE LRR DOMAIN

The protein sequences of the present resistance providing protein derived from reference genome, i.e., SEQ ID No. 5 and SEQ ID No. 6 contain the same protein domains. The domain length and order of the domains is conserved between the protein of the reference genome. In SEQ ID No. 5 and SEQ ID No. 6, however, several amino acid substitutions occurred. The skilled person is familiar with methods for the calculation of sequence similarity and sequence identity. Sequence similarity for an amino acid sequence is calculated using EMBOSS stretcher 6.6.0, using the EBLOSUM62 matrix with settings Gap open penalty: 12 and Gap extend penalty: 2.

Previously it was shown that the LRR protein domain has an important role in obtaining resistance against pathogens and comparing the LRR protein domain of the reference, SEQ ID No. 5 and SEQ ID No. 6 proteins resulted in #Identity: 316/333 (94.9%); #Similarity: 321/333 (96.4%); #Gaps: 0/333 (0.0%). Specific amino acids changes in the LRR domain are shown in Table 3.

Comparative genomics of Nanopore sequenced amplicons with the Augustus predicted gene and the splice variant showed that the LRR domain is still present in both versions.

TABLE 4
Mutations in the LRR domain.

| Amino acids position | | | |
|---|---|---|---|
| based on protein of | Amino acid substitution | | |
| reference genome | Reference | SEQ ID No. 6 | SEQ ID No. 5 |
| 574 | N | Y | Y |
| 591 | G | D | D |
| 688 | T | K | K |
| 697 | N | V | V |
| 698 | G | S | S |
| 714 | Y | N | N |
| 729 | R | K | K |
| 736 | Q | E | E |
| 742 | R | Q | Q |
| 774 | P | L | L |
| 778 | G | R | R |
| 838 | N | S | S |
| 842 | C | Y | Y |
| 847 | T | I | I |
| 861 | N | S | S |
| 867 | V | E | E |
| 892 | S | L | L |

### EXAMPLE 7. INTRODUCTION OF THE GENETIC FRAGMENTS PROVIDING PERONOSPORA FARINOSA RESISTANCE FROM SPINACIA TETRANDRA INTO SPINACIA OLERACEA WITH AGROBACTERIUM TUMEFACIENS

Transient transformation of plants with resistance genes by using Agrobacterium tumefaciens results in plants resistant to pathogens. To this end constructs harboring the genetically encoded resistances according to the present invention can be designed and obtained with molecular biology techniques. These constructs would be harboring:
(1) The genetic fragment encoding the resistance from a S. tetrandra accession referred to as SEQ ID No. 3;
(2) The genetic fragment encoding the resistance from a S. tetrandra accession referred to as SEQ ID No. 4.

Susceptible S. oleracea plants can be transformed with construct (1) or construct (2) using co-cultivation with A. tumefaciens. In addition positive and negative controls are included. Upon completed transformation, transformants can be subjected to a disease test using a P. farinosa isolate. It is expected that the transformants will be resistant to infection with P. farinosa, while the wild-type plants are still susceptible.

### EXAMPLE 8. VIRUS INDUCED GENE SILENCING EXPERIMENT (VIGS) TO SILENCE THE GENETICALLY ENCODED RESISTANCE FROM SPINACIA TETRANDRA

Tobacco rattle virus (TRV)-derived VIGS vectors have been abundantly described to study gene function in Arabidopsis thaliana, Nicotiana benthamiana, Lycopersicon esculentum and other plants. Prior to the experiment we received plasmids 0155-157 pTRV1 and 0158-160 pTRV2-MCS from the Arabidopsis Biological Resource Center.

To confirm that the genomic fragments from S. tetrandra, namely:
(i) The genetic fragment encoding the resistance from a S. tetrandra accession referred to as SEQ ID No. 3;
(ii) The genetic fragment encoding the resistance from a S. tetrandra accession referred to as SEQ ID No. 4.
   where these fragments are present in S. tetrandra plants according to this invention, are responsible for the observed resistance phenotype, a VIGS experiment can be carried out.

For this purpose VIGS constructs, targeting the earlier described genomic fragments (SEQ ID No. 3 and SEQ ID No. 4) were designed with pssRNAit. In addition, a positive VIGS control was used, and this positive control targets the (Phytoene Desaturase) PDS gene. The negative control is the empty vector.

Because of very high sequence identity between SEQ ID No. 3 and SEQ ID No. 4, the same SiRNA molecules can be used. Three sequences were targeted (VIGS1, VIGS2, VIGS3).

For PDS positive control, two sequences were targeted PDS1 and PDS 2, see below.

| | SIRNA | | No_of_off |
|---|---|---|---|
| | (anti-sense) | SIRNA (Sense) | targets |
| Vigs1 | UAUAAACCGG | GGCAUCGGCC | 0 |
| | GCCGAUGCCU | CGGUUUAUAA | |
| | U | G | |
| Vigs2 | AUAAACCGGG | AGGCAUCGGC | 0 |
| | CCGAUGCCUU | CCGGUUUAUA | |
| | C | A | |
| Vigs3 | CGAUGCAACC | CGCAGAAGGG | 0 |
| | CCUUCUGCGC | GUUGCAUCGA | |
| | U | C | |
| PDS 1 | UAUAAACCGG | GGCAUCGGCC | 0 |
| | GCCGAUGCCU | CGGUUUAUAA | |
| | U | G | |
| PDS 2 | CUAACGGGGU | GCUCCAGACA | 0 |
| | GUCUGGAGCC | CCCCGUUAGU | |
| | A | C | |

| | | | |
|---|---|---|---|
| SEQ ID | VIGS 1 | anti-sense SiRNA | UAUAAACCGG |
| No. 9 | | | GCCGAUGCCU |
| | | | U |
| SEQ ID | VIGS1 | sense SiRNA | GGCAUCGGCC |
| No. 10 | | | CGGUUUAUAA |
| | | | G |
| SEQ ID | VIGS 2 | anti-sense SiRNA | AUAAACCGGG |
| No. 11 | | | CCGAUGCCUU |
| | | | C |
| SEQ ID | VIGS 2 | sense SiRNA | AGGCAUCGGC |
| No. 12 | | | CCGGUUUAUA |
| | | | A |
| SEQ ID | VIGS 3 | anti-sense SiRNA | CGAUGCAACC |
| No. 13 | | | CCUUCUGCGC |
| | | | U |
| SEQ ID | VIGS 3 | sense SiRNA | CGCAGAAGGG |
| No. 14 | | | GUUGCAUCGA |
| | | | C |
| SEQ ID | PDS 1 | anti-sense SiRNA | UAUAAACCGG |
| No. 15 | | | GCCGAUGCCU |
| | | | U |
| SEQ ID | PDS 1 | sense SiRNA | CGCAGAAGGG |
| No. 16 | | | GUUGCAUCGA |
| SEQ ID | PDS 2 | anti-sense SiRNA | CUAACGGGGU |
| No. 17 | | | GUCUGGAGCC |
| | | | A |
| SEQ ID | PDS 2 | sense SiRNA | GCUCCAGACA |
| No. 18 | | | CCCCGUUAGU |
| | | | C |

Subsequently these fragments are obtained with molecular biology techniques, cloned into the VIGS plasmids above and transformed into Agrobacterium tumefaciens. Followed co-cultivation with A. tumefaciens with S. tetrandra plants according to this invention using, as specified below:

| VIGS construct target | Target plants for VIGS experiment |
|---|---|
| Fragment from S. tetrandra | Spinach plant comprising the |
| referred to as Seq 3 | S. tetrandra genomic fragments |
| Fragment from S. tetrandra | providing resistance |
| referred to as Seq 4 | |
| Phytoene Desaturase (PDS gene) | |

After the VIGS silencing, the obtained transformed plants can be subjected to a disease test using P. farinosa race 17. The non-transformed plants are still resistant to the pathogen, while it is expected that the plants where the VIGS experiments was successful, will become susceptible to P. farinosa.

Additionally, longer target sequences were designed and used. The longer fragments result in many small-RNAs and by this the change of successful silencing is significantly higher than using only one small-RNAs as input. Seq ID No 19 targets the resistance genes: the first and or the second genomic DNA from S. tetrandra. Seq ID No 20 targets the Phytoene Desaturase (control).

| Se- | | |
|---|---|---|
| quence | | |
| name | Targets | Sequence |
| SEQ ID | Resistance | ggatttcccgcagtgacactgacattatcg |
| No. | gene | aagcaacaatgaaggaacttgccaaacttt |
| 19 | | tcccagacgaaattgcagctgatgggagca |
| | | aggctaagatcctcaaatatcatgttgtca |
| | | agactccaaggtctgtttataagacagttc |
| | | cagactgtgagccttgtcggccactgcaaa |
| | | gatcaccactagaaggtttctatttatctg |
| | | gtgattacacaaagcaaaaatatttggctt |
| | | caatggaaggtgctgttttatctgggaagt |
| | | tttgtgcaca |
| SEQ ID | PDS | agatgaggacttggttttctttccatccct |
| No. | | tgaaaagcttgagctttggagtatgccaaa |
| 20 | | gttggaaggatggtggaaattagaatcaga |
| | | tatgggtgagacacgagaagtagcagggtt |
| | | tcaaacacattcatattcgttccatcacct |
| | | ttctcatctgttaattacattttgtgataa |
| | | tttgagaaattttcctctctgtccgaaact |
| | | ggaagaatcaaactcccaactacaggaaat |
| | | cagaaatctgaatcattcttttaccagtaa |
| | | gatcttcttttttattgtgtgtgtgtaaaa |
| | | ggttacatatttttatatatacctagtagc |
| | | tcctaactatctacaagtataactttgttc |
| | | atggtataagtgcagaagacgaagttgagt |
| | | ccctccaatccttgcaatttgaccttgaaa |
| | | ctatagaaattgcaacacataacttttctg |
| | | atgataataagatcggagaaggcatcggcc |
| | | cggtttataagggtatacttcctgatgggc |
| | | aagagatagaagttaagaggcttttaagga |
| | | actcgcgccaaagagatgcagagttcaaaa |
| | | atgaaattttgatattagccaaggtccggc |
| | | acaagaa |

1-5. (canceled)

6. A spinach plant which is resistant to the plant pathogen Peronospora farinosa, comprising in its genome:
a first genomic DNA fragment having the nucleic acid sequence of SEQ ID No. 1 or a nucleic acid sequence having at least 90% identity with SEQ ID No. 1; or
a second genomic DNA fragment having the nucleic acid sequence of SEQ ID No. 2 or a nucleic acid sequence having at least 90% identity with SEQ ID No. 2,
wherein the first and second genomic DNA fragment comprise a gene encoding a protein providing resistance against the plant pathogen Peronospora farinosa.

7. The spinach plant according to claim 6, wherein the plant is at least resistant to the plant pathogens Peronospora farinosa races Pfs 10 to Pfs 19.

8. The spinach plant according to claim 6, wherein the plant is further resistant to the plant pathogens Stemphylium vesicarium and/or CMV.

9. The spinach plant according to claim 6, wherein the resistance is obtained, is obtainable, or is from
deposit NCIMB 43993 for the first Spinacia tetrandra genomic fragment; or
deposit NCIMB 43994 for the second Spinacia tetrandra genomic fragment.

10. The spinach plant according to claim 6, wherein the plant produces seeds comprising at most 25% sharp seeds.

11. A seed or plant part produced by a spinach plant according to claim 6, wherein the seed or plant part comprises in its genome:
a first genomic DNA fragment having the nucleic acid sequence of SEQ ID No. 1 or a nucleic acid sequence having at least 90% identity with SEQ ID No. 1; or
a second genomic DNA fragment having the nucleic acid sequence of SEQ ID No. 2 or a nucleic acid sequence having at least 90% identity with SEQ ID No. 2,
wherein the first and second genomic DNA fragment comprise a gene encoding a protein providing resistance against the plant pathogen Peronospora farinosa.

12-13. (canceled)

14. A method for providing a spinach plant being resistant to the plant pathogen Peronospora farinosa, wherein the method comprises the step of introducing:
a first genomic DNA fragment having the nucleic acid sequence of SEQ ID No. 1 for a nucleic acid sequence having at least 90% identity with SEQ ID No. 1; or
a second genomic DNA fragment having the nucleic acid sequence of SEQ ID No. 2 or a nucleic acid sequence having at least 90% identity with SEQ ID No. 2,
into a pathogen Peronospora farinosa susceptible spinach plant.

15. The method for providing a spinach plant according to claim 14, wherein the method comprises the step of transformation using Agrobacterium and/or CRISPR Cas.

16. The method according to claim 14, wherein the first or a second genomic DNA fragment, is obtainable, is obtained, or is from the deposit number NCIMB 43993 and NCIMB 43994, respectively.

17. A method for identifying a spinach plant being resistant to Peronospora farinosa, wherein the method comprises:
isolating or providing plant material of the spinach plant; and
detecting in the plant material:
a nucleic acid having the sequence of SEQ ID No. 1 or a nucleic acid having at least 90% identity with SEQ ID No. 1; or
a nucleic acid having the sequence of SEQ ID No. 2 or a nucleic acid having at least 90% identity with SEQ ID No. 2; or
a protein having the amino acid sequence of SEQ ID No. 5 or a protein having at least 85% identity with SEQ ID No. 5; or
a protein having the amino acid sequence of SEQ ID No. 6 and/or SEQ ID No. 22, or a protein at least 85% identity with SEQ ID No. 6 and/or SEQ ID No. 22; or
a nucleic acid having the sequence of SEQ ID No. 3 or a nucleic acid having at least 90% identity with SEQ ID No. 3; or
a nucleic acid having the sequence of SEQ ID No. 4 and/or SEQ ID No. 21, or a nucleic acid having at least 90% identity with SEQ ID No. 4 and/or SEQ ID No. 21.

18. (canceled)

19. The spinach plant of claim 6, wherein
the first genomic DNA fragment comprises a gene encoding a first resistance protein providing resistance against the plant pathogen Peronospora farinosa, wherein the first resistance protein comprises the amino acid sequence of SEQ ID No. 5 or an amino acid sequence having at least 85% identity with SEQ ID No. 5; or
the second genomic DNA fragment comprises a gene encoding a second resistance protein providing resistance against the plant pathogen Peronospora farinosa, wherein the second resistance protein comprises the amino acid sequence of SEQ ID No. 6 and/or SEQ ID No. 22, or a sequence having at least 85% identity with SEQ ID No. 6 and/or SEQ ID No. 22.

20. The spinach plant of claim 6, wherein
the first genomic DNA fragment comprises the nucleic acid sequence of SEQ ID No. 3 or a sequence having at least 90% identity with SEQ ID No. 3; or
the second genomic DNA fragment comprises the nucleic acid sequence of SEQ ID No. 4 and/or SEQ ID No. 21, or a sequence having at least 90% identity with SEQ ID No. 4 and/or SEQ ID No. 21.
